(19) United States
(12) Patent Application Publication
(10) Pub. No.: US 2005/0065213 A1
(43) Pub. Date: Mar. 24, 2005

(54) Combretastatin a-4 derivatives having antineoplastic activity

(76) Inventors: Nicholas James Lawrence, Bristol (GB); John Anthony Hadfield, Salford (GB); Alan Thomson McGown, Manchester (GB); John Butler, Manchester (GB); Sylvie Ducki, Salford (GB); David Rennison, Auckland (NZ); Meiki Woo, Manchester (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/494,865

(22) PCT Filed: Nov. 8, 2002
(86) PCT No.: PCT/GB02/05055

(30) Foreign Application Priority Data
Nov. 8, 2001 (GB) 0126889.5

(51) Int. Cl. A61K 31/221; A61K 31/165; A61K 31/137

(52) U.S. Cl. 514/550; 514/622; 514/649; 514/679; 560/145; 564/169; 564/390

(57) ABSTRACT

Compounds are disclosed that are designed to mimic the activity of combretastatin A-4 based on chalcone, aurone, or indanone structures, or involving benzoquinone or quinone rings. The anti-cancer activity of exemplified compounds is demonstrated in a range of in vitro and in vivo assays.

### FIELD OF THE INVENTION

The present invention relates to compounds and their uses, and more particularly to chalcone, indanone, aurone and quinone compounds which are structurally related to combretastatin A-4 and their possible use as anticancer compounds. The present invention also of these and other compounds in the treatment of cancer.

### BACKGROUND OF THE INVENTION

The stilbene cis-combretastatin A-4 (hereafter referred to as “CA-4”), isolated from the African bush willow, Combretum caffrum shows exciting potential as an anticancer agent, binding strongly to tubulin and displaying potent and selective toxicity toward tumour vasculature (U.S. Pat. No. 4,996,237. cis-combretastatin A-4 is able to inhibit cell growth at low concentrations (IC₅₀, P388 murine leukaemia cell line 2.6 nM). The potency of trans-combretastatin A-4 is much lower and inhibits cell growth in the μM range. Arguably, it is the ability of cis-combretastatin A-4 to destroy tumour blood vessels, effectively starving tumours of nutrients, which makes them such exciting molecules. Tumour vasculature and the formation of neovasculature were first identified as a target for cancer therapy by Judah Folkman some 30 years ago. The work of Folkman and others has clearly identified angiogenesis and blood supply as necessary requirements for primary tumour growth, invasiveness and metastasis. It is now becoming clear that the selective destruction of tumour vasculature will have a significant impact on the clinical treatment of cancer. Angiogenesis is subject to a complex process of regulation and thereby offers a multitude of molecular targets for drug design.

We have previously investigated the tubulin-binding properties of agents related to CA-4 and colchicine and as part of this effort, we have designed many related compounds that behave in a similar fashion to CA-4 (Ducki et al, Bioorg. Med. Chem. Lett., 1998, 8, 1051; Zhao et al, Eur. J. Nuc. Medicine, 1999, 26, 231; Aleksandrzak et al, Anti-Cancer Drugs, 1998, 9, 545).

Considerable effort has been expended in an attempt to synthesis and characterise compounds suitable for use in anti-tumour therapies. By way of example, U.S. Pat. No. 6,071,930 describes the synthesis of a series of 2-aryl-1,8-naphthyridiones, which have amino analogues of cytotoxic antimitotic flavonoids. The authors found that many of these compounds were cytotoxic and possessed activity against tubulin polymerisation and colchicine binding.

EP 0 288 794 A2 describes the use of a number of chalcone derivatives bearing either —NR₂ or —NHCOR groups (where R is C₁-C₄ alkyl), for treating growth of tumour tissues.

Clark et al, in the international patent application WO00/35865, disclose natural product derivatives and derivatives of known tubulin-binding compounds in which a (poly)fluorobenzene, fluoropyridine, or fluoronitrophenyl moiety is incorporated or added to the structure. These derivatives can be used as antimitotic agents.

Ring-contracted analogues of the antitumour agent etoposide have been prepared by Klein et al. and the cytotoxicity of the derivatives towards several tumour cell lines has also been reported.

Beutler et al have screened over 70 known flavones for cytotoxicity in the NCI in vitro 60-cell line human tumour screen. The tests demonstrated that flavones which are not substituted at the carbon alpha to the ketone have a minimal cytotoxicity.

Compounds isolated from leaf and stem extracts of Uvaria hamiltonii were tested for activity in a 9 KB cytotoxicity assay. In contrast to the studies of Beutler et al., flavanones and aurones were found to be inactive, and chalcone compounds demonstrated only weak activity.

Despite ongoing attempts to synthesis compounds with anti-tumour activity, it remains a problem in the art in designing effective compounds.

### SUMMARY OF THE INVENTION

At its broadest, the present invention provides new potential anti-cancer compounds, structurally related to combretastatin A-4, and their use, along with related compounds, in the treatment of cancer and other conditions involving abnormal proliferation of vasculature.

The compounds of the present invention represent a new range of potential anti-tumour drugs.

In some embodiments, the compounds of the present invention are based on the chalcone structure and are either substituted chalcones or conformationally restricted analogues of chalcones, all being related to the CA-4 structure.

The synthesis of new compounds is disclosed herein, together with experiments demonstrating their activity in cytotoxicity (IC₅₀) assays against the K562 cell line and supporting their use as anticancer compounds and prodrugs.

Accordingly, in a first aspect, the present invention provides a family of anti-cancer compounds based on chalcone, indanone, aurone and quinone structures, including fluorinated, nitro, amine and phosphate substituted analogues. The family of compounds includes structures where the ketone has been reduced to an alcohol, alkene or alkane.

Thus, in this aspect, the present invention provides compounds represented by the structural formula (I): 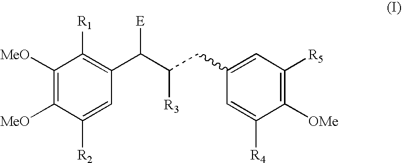
wherein:
E represents an oxo (═O) or a hydroxyl (—OH); the dashed line indicates that a single or double bond may be present;
the zig-zag line indicates that the compound can be either the E or Z isomer;
R₃ is H, alkyl, CH₂NH₂, CH₂NHalkyl, CH₂OH, CH₂N(alkyl)₂, CH₂NH(C═O)alkyl, CH₂NH(C═O)aryl; and
R₄ is H, halogen, NH(alkyl), N(alkyl)₂, NH(C═O)alkyl, NH(C═O)aryl, or a Boc-ester group represented by: 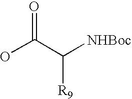
   wherein R₉ is alkyl, CH₂Ph where Ph is a substituted or substituted phenyl group, or an amino acid side chain; and further wherein
when E is an oxo (═O) group and the dashed line represents a single bond,
R₁ is H; R₂ is alkoxy; R₄ is H; and R₅ is OH; or
when E is an oxo (═O) group and the dashed line represents a double bond,
R₁ is H; R₂ is alkoxy; R₄ is H or halogen; and
R₅ is H or halogen; or
R₄ is H; and R₅ is NH₂, NO₂, halogen or OPO₃ (R₆)₂; where R₆ is H, CH₂Ph or a metal cation; or
R₁ is alkoxy; R₂ is H; R₄ is H or halogen; and
R₅ is halogen or OH; or
when E is a hydroxyl (—OH) group and the dashed line represents a single or double bond,
R₁ is H; R₂ is alkoxy; R₃ is methyl; R₄ is H; and R₅ is OH;
   or a salt or derivative thereof.

In all aspects of the invention, preferably, the substituents are chosen according to the following list of preferred groups.

Preferably, alkyl or alkoxy substituents are substituted or unsubstituted, branched or unbranched C₁₋₁₀ alkyl or alkoxy groups. Preferred alkyl substituents are methyl or ethyl. Preferred alkoxy substituents are methoxy, ethoxy or propoxy.

Halogen substituents can be fluorine, chlorine, bromine or iodine, and are preferably fluorine.

As used herein, preferably R and R′ are substituted or unsubstituted, branched or unbranched C₁₋₁₀ alkyl groups or aryl or heteroaryl groups.

As used herein, the Boc-ester group wherein X is a group represented by: 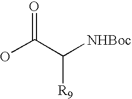
wherein R₉ is alkyl, CH₂Ph where Ph is a substituted or substituted phenyl group, or an amino acid side chain, and Boc represents a t-butoxycarbonyl group. The amino acid ester side chain may include a naturally occurring or synthetic amino acid, in either the D or L-isoform. Examples of compounds of the aspect of the invention include those where the amino acid is Phe, Ile, Gly, Trp, Met, Leu, Ala, His, Pro, D-Met, D-Trp, or Tyr, e.g. when the amino acid is Phe, R₉ group is —CH₂Ph etc. Further information on the preparation of Boc esters is provided in WO 02/50007.

In a preferred embodiment, the present invention provides a compound represented by formula (I) where:
E is an oxo (═O) group; the dashed line represents a single bond; R₁ is H; R₂ is OMe; R₃ is H; R₄ is H; and R₅ is OH (MW57);
E is an oxo (═O) group; the dashed line represents a single bond; R₁ is H; R₂ is OMe; R₃ is Me; R₄ is H; and R₅ is OH (MW71);
E is an oxo (═O) group; the dashed line represents a double bond; R₁ is H; R₂ is OMe; R₃ is H; R₄ is H; and R₅ is NH₂ (MW65);
E is an oxo (═O) group; the dashed line represents a double bond; R₁ is H; R₂ is OMe; R₃ is H; R₄ is H; and R₅ is NO₂ (MW47);
E is an oxo (═O) group; the dashed line represents a double bond; the compound is the E isomer; R₁ is H; R₂ is OMe; R₃ is Me; R₄ is H; and R₅ is NO₂ (MW68);
E is an oxo (═O) group; the dashed line represents a double bond; the compound is the Z isomer; R₁ is H; R₂ is OMe; R₃ is Me; R₄ is H; and R₅ is NO₂ (MW69);
E is an oxo (═O) group; the dashed line represent a double bond; R₁ is H; R₂ is OMe; R₃ is H; R₄ is H; and R₅ is F (DR2);
E is an oxo (═O) group; the dashed line represent a double bond; R₁ is H; R₂ is OMe; R₃ is H; R₄ is F; and R₅ is F (DR3);
E is an oxo (═O) group; the dashed line represent a double bond; R₁ is H; R₂ is OMe; R₃ is Me; R₄ is H; and R₅ is F (DR5);
E is an oxo (═O) group; the dashed line represent a double bond; R₁ is H; R₂ is OMe; R₃ is Me; R₄ is F; and R₅ is F (DR6);
E is an oxo (═O) group; the dashed line represent a double bond; R₁ is OMe; R₂ is H; R₃ is H; R₄ is H; and R₅ is OH (DR8);
E is an oxo (═O) group; the dashed line represent a double bond; R₁ is OMe; R₂ is H; R₃ is H; R₄ is H; and R₅ is F (DR9);
E is an oxo (═O) group; the dashed line represent a double bond; R₁ is OMe; R₂ is H; R₃ is H; R₄ is F; and R₅ is F (DR10);
E is an oxo (═O) group; the dashed line represent a double bond; R₁ is H; R₂ is OMe; R₃ is Me; R₄ is H; and R₅ is OPO₃(R₆)₂ wherein R₆ is CH₂Ph (DR53);
E is an oxo (═O) group; the dashed line represent a double bond; R₁ is H; R₂ is OMe; R₃ is H; R₄ is H; and R₅ is OPO₃(R₆)₂ wherein R₆ is CH₂Ph (DR54);
E is an oxo (═O) group; the dashed line represent a double bond; R₁ is H; R₂ is OMe; R₃ is Me; R₄ is H; and R₅ is OPO₃(R₆)₂ wherein R₆ is H (DR55);
E is an oxo (═O) group; the dashed line represent a double bond; R₁ is H; R₂ is OMe; R₃ is H; R₄ is H; and R₅ is OPO₃ (R₆)₂ wherein R₆ is H (DR56);
E is an oxo (═O) group; the dashed line represent a double bond; R₁ is H; R₂ is OMe; R₃ is H; R₄ is H; and R₅ is OPO₃(R₆)₂ wherein R₆ is H (SD173a);
E is an oxo (═O) group; the dashed line represent a double bond; R₁ is H; R₂ is OMe; R₃ is H; R₄ is H; and R₅ is OPO₃(R₆)₂ wherein R₆ is Na (SD174a);
E is an oxo (═O) group; the dashed line represent a double bond; R₁ is H; R₂ is OMe; R₃ is Me; R₄ is H; and R₅ is OPO₃(R₆)₂ wherein R₆ is Na (SD174b);
E is a hydroxyl (—OH) group; the dashed line represents a single bond; R₁ is H; R₂ is OMe; R₃ is Me; R₄ is H; and R₅ is OH (MW72);
E is a hydroxyl (—OH) group; the dashed line represents a single bond; R₁ is H; R₂ is OMe; R₃ is H; R₄ is H; and R₅ is OH (MW58);
E is a hydroxyl (—OH) group; the dashed line represents a double bond; R₁ is H; R₂ is OMe; R₃ is H; R₄ is H; and R₅ is OH (MW50);
E is a hydroxyl (—OH) group; the dashed line represents a double bond; R₁ is H; R₂ is OMe; R₃ is Me; R₄ is H; and R₅ is OH (MW70);

In this aspect, the present invention provides a further family of compounds based on the chalcone structure, including fluorinated analogues.

Accordingly, the present invention provides compounds represented by the structural formula (Ia): 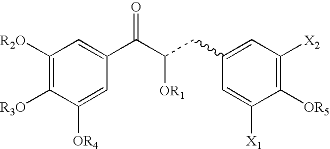
wherein:
the dashed line indicates that a single or double bond may be present;
the zig-zag line indicates that the compound can be either the E or Z isomer;
R₁ is alkyl; R₂, R₃, R₄ and R₅ are independently selected from H or alkyl; X₁ and X₂ are independently selected from H, OH, nitro, amino, aryl, heteroaryl, alkyl, alkoxy, CHO, COR, halogen, haloalkyl, NH₂, NHR, NRR′, SR, CONH₂, CONHR, CONHRR′, O—P═O(OR)₂, O-aryl, O-heteroaryl, O-ester or a Boc-ester group represented by: 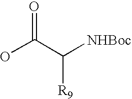
   wherein R₉ is alkyl, CH₂Ph where Ph is a substituted or substituted phenyl group, or an amino acid side chain;
   or a salt or derivative thereof.

In a preferred embodiment, the present invention provides: a compound represented by formula (Ia) when
the dashed line represent a double bond; R₁ is Me; R₂, R₃ and R₄ are Me; R₅ is Me; X₁ is H; and X₂ is OH (DR13); or
the dashed line represent a double bond; R₁ is Me; R₂, R₃ and R₄ are Me; R₅ is Me; X₁ is H; and X₂ is F (DR14); or
the dashed line represent a double bond; R₁ is Me; R₂, R₃ and R₄ are Me; R₅ is Me; X₁ and X₂ are F (DR15); or
the dashed line represent a double bond; R₁ is Et; R₂, R₃ and R₄ are Me; R₅ is Me; X₁ is H; and X₂ is OH (DR16); or
the dashed line represent a double bond; R₁ is Et; R₂, R₃ and R₄ are Me; R₅ is Me; X₁ is H; and X₂ is F (DR17); or
the dashed line represent a double bond; R₁ is Et; R₂, R₃ and R₄ are Me; R₅ is Me; X₁ and X₂ are F (DR18); or
the dashed line represent a double bond; R₁ is Pr; R₂, R₃ and R₄ are Me; R₅ is Me; X₁ is H; and X₂ is OH (DR19); or
the dashed line represent a double bond; R₁ is Pr; R₂, R₃ and R₄ are Me; R₅ is Me; X₁ is H; and X₂ is F (DR20); or
the dashed line represent a double bond; R₁ is Pr; R₂, R₃ and R₄ are Me; R₅ is Me; X₁ is F; and X₂ is F (DR21);

In this aspect, the present invention provides a family of compounds based on the indanone structure, including reduced forms of the ketone, and fluorinated analogues.

Accordingly, the present invention provides compounds represented by the structural formula (II): 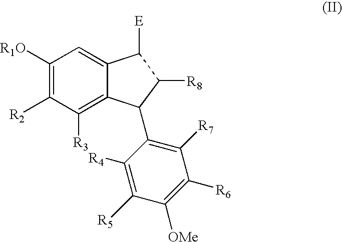
wherein:
E represents an oxo (═O), hydroxyl (—OH) or a hydrogen atom;
the dashed line in the structure indicates that a single or double bond may be present; and
R₈ is hydrogen, alkyl, aryl, CH₂NH₂, CH₂NHalkyl or CH₂N(alkyl)₂; and wherein
when E is an oxo (═O) group and the dashed line represents a single bond,
R₁ is alkyl or H; R₂ is alkoxy or H; R₃ is alkoxy or H; and R₄ is H; R₅ is H, O(P═O) (OR)₂ or Boc-ester;
R₆ is NO₂, NH₂, H, OH, halogen, NHMe, NHMe₂, NH(C═O)alkyl or NH(C═O)aryl; and R₇ is H; or
R₄ is H; R₅ is halogen, O(P═O) (OR)₂ or Boc-ester;
R₆ is OH, halogen, NHMe, NHMe₂, NH(C═O)alkyl or NH(C═O)aryl; and R₁ is H; or
R₄ is alkoxy; R₅ is H, O(P═O) (OR)₂ or Boc-ester;
R₆ is H, NHMe, NHMe₂, NH(C═O)alkyl or NH(C═O)aryl; and R₇ is alkoxy; or
when E is a hydroxyl (—OH) group and the dashed line represents a single bond,
R₁ is alkyl; R₂ is H or alkoxy; R₃ is alkoxy; R₄ is H; R₅ is alkoxy, halogen, O(P═O) (OR)₂ or Boc-ester;
R₆ is H, NO₂, NH₂, OH, halogen, NHMe, NHMe₂, NH(C═O)alkyl or NH(C═O)aryl; and R₇ is H; or
when E is a hydrogen atom and the dashed line represents a double bond,
R₁ is Me; R₂ is alkoxy; R₃ is alkoxy; R₄ is H; R₅ is H, O(P═O) (OR)₂ or Boc-ester;
R₆ is NO₂, NH₂, NHMe, NHMe₂, NH(C═O)alkyl or NH(C═O)aryl; and R₇ is H;
   wherein the Boc-ester is a group represented by: 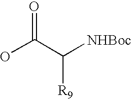
   wherein R₉ is alkyl, CH₂Ph where Ph is a substituted or substituted phenyl group, or an amino acid side chain; or
a compound represented by structural formula (IIa), 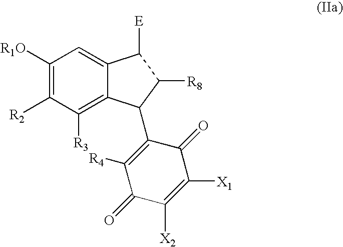
   wherein:
E, R₁, R₂, R₇ and R₈ are as defined above; and
X₁ and X₂ are independently selected from H, OH, nitro, amino, aryl, heteroaryl, alkyl, alkoxy, CHO, COR, halogen, haloalkyl, NH₂, NHR, NRR′, SR, CONH₂, CONHR, CONHRR′, O-aryl, O-heteroaryl or O-ester; or
or salts and derivatives of compounds II or IIa.

In a preferred embodiment, the present invention provides: a compound represented by formula (II) when E is an oxo (═O) group; the dashed line represents a single bond; R₁ is Me; R₂ is OMe; R₃ is OMe; R₄ is H; R₅ is H; R₆ is NO₂; R₇ is H (MW73); or
E is an oxo (═O) group; the dashed line represents a single bond; R₁ is Me; R₂ is OMe; R₃ is OMe; R₄ is H; R₅ is H; R₆ is NH₂; and R₇ is H (MW74); or
E is an oxo (═O) group; the dashed line represents a single bond; R₁ is Me; R₂ is OMe; R₃ is OMe; R₄ is H; R₅ is H; R₆ is H; and R₇ is H (DM23); or
E is an oxo (═O) group; the dashed line represents a single bond; R₁ is Me; R₂ is OMe; R₃ is OMe; R₄ is H; R₅ is H; R₆ is OH; and R₇ is H (DM13); or
E is an oxo (═O) group; the dashed line represents a single bond; R₁ is Me; R₂ is H; R₃ is OMe; R₄ is H; R₅ is H; R₆ is OH; and R₇ is H (DM25); or
E is an oxo (═O) group; the dashed line represents a single bond; R₁ is Me; R₂ is OH; R₃ is H; R₄ is OMe; R₅ is H; R₆ is H; and R₇ is OMe (DM26); or
E is an oxo (═O) group; the dashed line represents a single bond; R₁ is Me; R₂ is OMe; R₃ is OMe; R₄ is H; R₅ is H; R₆ is F; and R₇ is H (DR59); or
E is an oxo (═O) group; the dashed line represents a single bond; R₁ is Me; R₂ is OMe; R₃ is OMe; R₄ is H; R₅ is F; R₆ is F; and R₇ is H (DR61); or
E is a hydroxyl (—OH) group; the dashed line represents a single bond; R₁ is Me; R₂ is OMe; R₃ is OMe; R₄ is H; R₅ is H; R₆ is NO₂; R₇ is H (MW76); or
E is a hydroxyl (—OH) group; the dashed line represents a single bond; R₁ is Me; R₂ is OMe; R₃ is OMe; R₄ is H; R₅ is H; R₆ is NH₂; and R₇ is H (MW77); or
E is a hydroxyl (—OH) group; the dashed line represents a single bond; R₁ is Me; R₂ is OMe; R₃ is OMe; R₄ is H; R₅ is H; R₆ is H; and R₇ is H (DM28); or
E is a hydroxyl (—OH) group; the dashed line represents a single bond; R₁ is Me; R₂ is OMe; R₃ is OMe; R₄ is H; R₅ is H; R₆ is OH; and R₇ is H (DM29); or
E is a hydroxyl (—OH) group; the dashed line represents a single bond; R₁ is Me; R₂ is H; R₃ is OMe; R₄ is H; R₅ is H; R₆ is OH; and R₇ is H (DM31); or
E is a hydroxyl (—OH) group; the dashed line represents a single bond; R₁ is Me; R₂ is OMe; R₃ is OMe; R₄ is H; R₅ is H; R₆ is F; and R₇ is H (DR60); or
E is a hydroxyl (—OH) group; the dashed line represents a single bond; R₁ is Me; R₂ is OMe; R₃ is OMe; R₄ is H; R₅ is F; R₆ is F; and R₇ is H (DR62); or
E is a hydrogen atom; the dashed line represents a single bond; R₁ is Me; R₂ is OMe; R₃ is OMe; R₄ is H; R₅ is H; R₆ is NO₂; and R₇ is H (MW75); or
E is a hydrogen atom; the dashed line represents a double bond; R₁ is Me; R₂ is OMe; R₃ is OMe; R₄ is H; R₅ is H; R₆ is NO₂; and R₇ is H (MW81); or
E is a hydrogen atom; the dashed line represents a single bond; R₁ is Me; R₂ is OMe; R₃ is OMe; R₄ is H; R₅ is H; R₆ is NH₂; and R₇ is H (MW82); or

In this aspect, the present invention provides a family of compounds based on the aurone structure, including fluorinated analogues.

Accordingly, the present invention provides compounds represented by the structural formula (III): 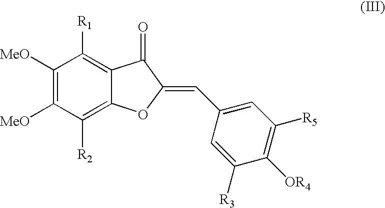
wherein:
R₁ is H or alkoxy; R₂ is H or alkoxy; R₃ is H or halogen;
R₄ is H or alkyl; and R₅ is H, OH, halogen, O(P═O) (OR)₂ or
   a Boc-ester group represented by: 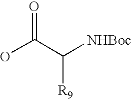
   wherein R₉ is alkyl, CH₂Ph where Ph is a substituted or substituted phenyl group, or an amino acid side chain; or a salt or derivative thereof.

In a preferred embodiment, the present invention provides: a compound represented by formula (III) when
R₁ is OMe; R₂ is H; R₃ is H; R₄ is Me; R₅ is H (DR22); or
R₁ is OMe; R₂ is H; R₃ is H; R₄ is Me; R₅ is OH (DR23); or
R₁ is OMe; R₂ is H; R₃ is H; R₄ is Me; R₅ is F (DR24); or
R₁ is OMe; R₂ is H; R₃ is F; R₄ is Me; R₅ is F (DR25); or
R₁ is H; R₂ is OMe; R₃ is H; R₄ is Me; R₅ is H (DR26); or
R₁ is H; R₂ is OMe; R₃ is H; R₄ is Me; R₅ is OH (DR27); or
R₁ is H; R₂ is OMe; R₃ is H; R₄ is Me; R₅ is F (DR28); or
R₁ is H; R₂ is OMe; R₃ is F; R₄ is Me; R₅ is F (DR29); or
R₁ is H; R₂ is OMe; R₃ is H; R₄ is H; R₅ is OH (DR31).

In a further aspect, the present invention provides a family of compounds with a substituted or unsubstituted benzoquinone/quinone ring.

Accordingly, the present invention provides compounds represented by the structural formula (IV): 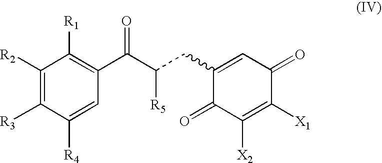
wherein:
the dashed line indicates that a single or double bond may be present;
the zig-zag line indicates that the compound can be either the E or Z isomer; and
R₁, R₂, R₃ and R₄ are independently selected from H or alkoxy;
R₅ is hydrogen, alkyl, alkoxy or O-aryl; and
X₁ and X₂ are independently selected from H, OH, nitro, amino, aryl, heteroaryl, alkyl, alkoxy, CHO, COR, halogen, haloalkyl, NH₂, NHR, NRR′, SR, CONH₂. CONHR, CONHRR′, O-aryl, O-heteroaryl or O-ester;
or a salt or derivative thereof.

In a preferred embodiment, the present invention provides: a compound represented by the formula (IV) when
the dashed line represents a double bond; R₁ is H; R₂ is OMe; R₃ is OMe; R₄ is OMe, X₁ is OMe, and X₂ is H.

In a further aspect, the present invention provides a pharmaceutical composition, comprising one or more compounds as defined above, their salts or a mixture of both.

The use of amine functional groups in the compounds means that they can form salts and by variation of the salts (counterion, etc), the solubility properties of the compound can be altered. Variation of the salt (counterion, etc) represents another method of directing the activity of the compound, and forms part of the present invention.

The compounds disclosed here have been prepared and tested as racemic mixtures. It is expected that the pure enantiomers are likely to posses altered activity, one enantiomer being significantly more active than the other. The compounds of the invention will bind to proteins in the course of their action and therefore the chirality of the compound is likely to be important in determining their effectiveness.

Therefore, the individual enantiomers of compounds disclosed herein also form part of the present invention.

In a further aspect, the present invention provides a compound as defined above for use in a method of medical treatment.

In a further aspect, the present invention provides the use of a compound as defined above for the preparation of a medicament for the treatment of cancer or another condition involving abnormal proliferation of vasculature. Examples of these conditions include diabetic retinopathy, psoriasis and endometriosis.

In addition, the present invention provides compounds represented by the structural formulae (V) and (Va) and their use in a method of medical treatment: 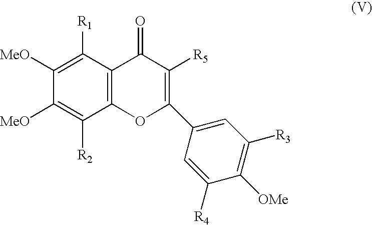
wherein:
R₁ or R₂ is alkoxy and the other is H;
R₃ and R₄ are different and are hydrogen, halogen, OH,
O(P═O)(OR)₂ or Boc-ester;
R₅ is aryl, alkyl or O-alkyl;
   wherein the Boc-ester group represented by: 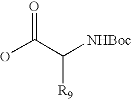
   wherein R₉ is alkyl, CH₂Ph where Ph is a substituted or substituted phenyl group, or an amino acid side chain; or
a compound of represented by structural formula (Va) in which: 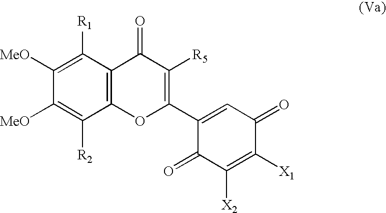
wherein:
R₁, R₂ and R₅ are defined as above;
X₁ and X₂ are independently selected from H, OH, nitro, amino, aryl, heteroaryl, alkyl, alkoxy, CHO, COR, halogen, haloalkyl, NH₂, NHR, NRR′, SR, CONH₂, CONHR, CONHRR′, O-aryl, O-heteroaryl or O-ester; or
or salts and derivatives of compounds V or Va.

In a preferred embodiment, the present invention provides: a compound used in a method of medical treatment, represented by formula (V) when
R₁ is OMe; R₂ is H; R₃ is OH; and R₄ is H; or
R₁ is OMe; R₂ is H; R₃ is F; and R₄ is H; or
R₁ is H; R₂ is OMe; R₃ is OH; and R₄ is H; or
R₁ is OMe; R₂ is H; R₃ is F; and R₄ is H.

In a further aspect, the present invention provides the use of a compound as defined above for the preparation of a medicament for the treatment of cancer or another condition involving abnormal proliferation of vasculature. Examples of these conditions include diabetic retinopathy, psoriasis and endometriosis.

Embodiments of the present invention will now be described by way of example and not limitation with reference to the accompanying figures.

### BRIEF DESCRIPTION OF THE FIGURES

FIG. 1 shows the base catalysed condensation of an aldehyde and acetophenone to form chalcone structures.

FIG. 2 shows the Knoevenagel-like condensation of substituted acetophenone and benzaldehyde.

FIG. 3 shows the trifluoroacetic acid catalysed ring closure of chalcones to form indanones.

FIG. 4 shows the base catalysed formation of aurones.

FIG. 5 shows the results of treating H460 xenograft mice with compound DR5 compared to control.

FIG. 6 shows the results of treating H460 xenograft mice with compound DR5 in combination with X-ray treatment compared to control.

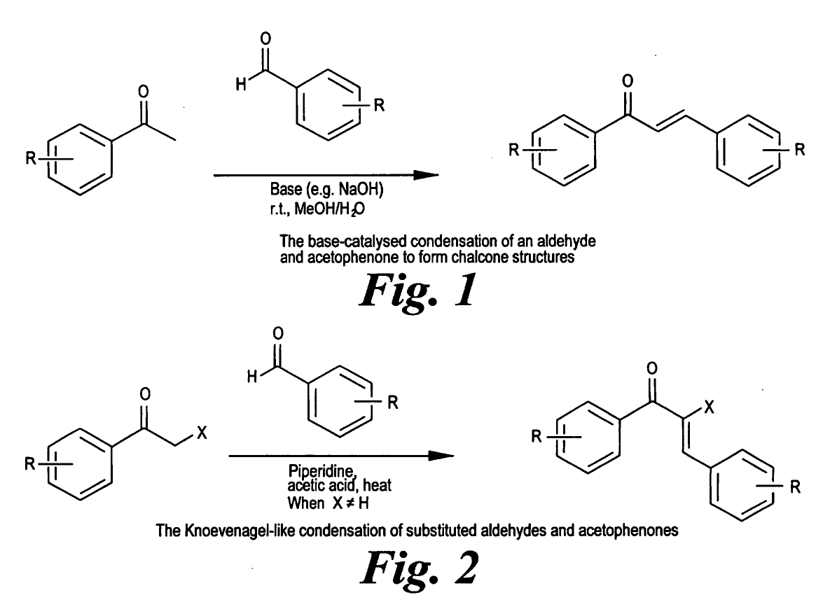

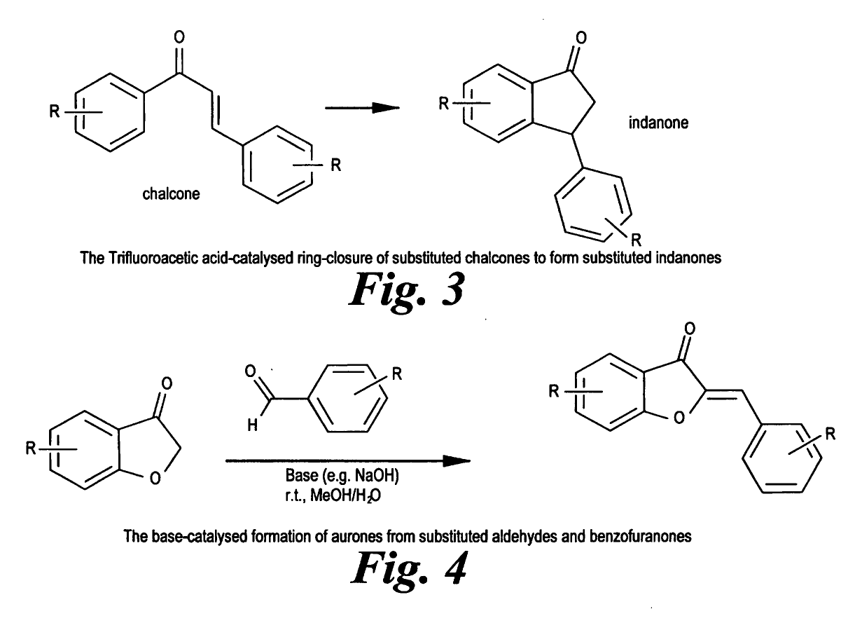

### DETAILED DESCRIPTION

Pharmaceutical Compositions

The compounds of the invention may be derivatised in various ways. As used herein “derivatives” of the compounds includes salts, esters such as in vivo hydrolysable esters, free acids or bases, hydrates, prodrugs or coupling partners. In the case of compounds which are combretastatin or analogues thereof, preferably the derivatives are soluble in water and/or saline or can be hydrolysed to provide physiologically active agents.

Examples in the prior art of salts or prodrugs of cis-combretastatin A-4 focus on forming salts or derivatives at the phenolic hydroxyl group of combretastatin. These include sodium phosphate salts, sodium and potassium salts (U.S. Pat. No. 5,561,122), lithium, caesium, magnesium, calcium, manganese and zinc salts of cis-combretastatin A-4, and ammonium cation salts with imidazole, morpholine, piperazine, piperidine, pyrazole, pyridine, adenosine, cinchonine, glucosamine, quinine, quinidine, tetracycline and verapamil (WO99/35150).

Without wishing to be bound by any particular explanation, the inventors believe that compounds of the invention including quinone and benzoquinone groups are activated in vivo by enzymes such as DT-diaphorase, reducing or hydrolysing the compounds to produce active forms of them. Thus, compounds including the quinone or benzoquinone groups can be regarded as prodrugs for active forms of the compounds, see also WO 02/50007.

Salts of the compounds of the invention are preferably physiologically well tolerated and non toxic. Many examples of salts are known to those skilled in the art. Compounds having acidic groups, can form salts with alkaline or alkaline earth metals such as Na, K, Mg and Ca, and with organic amines such as triethylamine and Tris (2-hydroxyethyl)amine. Salts can be formed between compounds with basic groups, e.g. amines, with inorganic acids such as hydrochloric acid, phosphoric acid or sulfuric acid, or organic acids such as acetic acid, citric acid, benzoic acid, fumaric acid, or tartaric acid. Compounds having both acidic and basic groups can form internal salts.

Esters can be formed between hydroxyl or carboxylic acid groups present in the compound and an appropriate carboxylic acid or alcohol reaction partner, using techniques well known in the art. Examples of esters include those formed between the phenolic hydroxyl of the substituted stilbenes and carboxylic acids, hemisuccinic acid esters, phosphate esters, BOC esters, sulphate esters and selenate esters.

Derivatives which as prodrugs of the compounds are convertible in vivo or in vitro into one of the parent compounds. Typically, at least one of the biological activities of compound will be reduced in the prodrug form of the compound, and can be activated by conversion of the prodrug to release the compound or a metabolite of it. Examples of prodrugs include phosphate derivatives.

Other derivatives include coupling partners of the compounds in which the compounds is linked to a coupling partner, e.g. by being chemically coupled to the compound or physically associated with it. Examples of coupling partners include a label or reporter molecule, a supporting substrate, a carrier or transport molecule, an effector, a drug, an antibody or an inhibitor. Coupling partners can be covalently linked to compounds of the invention via an appropriate functional group on the compound such as a hydroxyl group, a carboxyl group or an amino group.

The compounds described herein or their derivatives can be formulated in pharmaceutical compositions, and administered to patients in a variety of forms, in particular to treat conditions which are ameliorated by the activation of the compound.

Pharmaceutical compositions for oral administration may be in tablet, capsule, powder, cream, liquid form or encapsulated by liposomes. A tablet may include a solid carrier such as gelatin or an adjuvant or an inert diluent. Liquid pharmaceutical compositions generally include a liquid carrier such as water, petroleum, animal or vegetable oils, mineral oil or synthetic oil. Physiological saline solution, or glycols such as ethylene glycol, propylene glycol or polyethylene glycol may be included. Such compositions and preparations generally contain at least 0.1 wt % of the compound.

Parental administration includes administration by the following routes: intravenous, cutaneous or subcutaneous, nasal, intramuscular, intraocular, transepithelial, intraperitoneal and topical (including dermal, ocular, rectal, nasal, inhalation and aerosol), and rectal systemic routes. For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, solutions of the compounds or a derivative thereof, e.g. in physiological saline, a dispersion prepared with glycerol, liquid polyethylene glycol or oils.

In addition to one or more of the compounds, optionally in combination with other active ingredient, the compositions can comprise one or more of a pharmaceutically acceptable excipient, carrier, buffer, stabiliser, isotonicizing agent, preservative or anti-oxidant or other materials well known to those skilled in the art. Such materials should be non-toxic and should not interfere with the efficacy of the active ingredient. The precise nature of the carrier or other material may depend on the route of administration, e.g. orally or parentally.

Liquid pharmaceutical compositions are typically formulated to have a pH between about 3.0 and 9.0, more preferably between about 4.5 and 8.5 and still more preferably between about 5.0 and 8.0. The pH of a composition can be maintained by the use of a buffer such as acetate, citrate, phosphate, succinate, Tris or histidine, typically employed in the range from about 1 mM to 50 mM. The pH of compositions can otherwise be adjusted by using physiologically acceptable acids or bases.

Preservatives are generally included in pharmaceutical compositions to retard microbial growth, extending the shelf life of the compositions and allowing multiple use packaging. Examples of preservatives include phenol, meta-cresol, benzyl alcohol, para-hydroxybenzoic acid and its esters, methyl paraben, propyl paraben, benzalconium chloride and benzethonium chloride. Preservatives are typically employed in the range of about 0.1 to 1.0% (w/v).

Preferably, the pharmaceutically compositions are given to an individual in a “prophylactically effective amount” or a “therapeutically effective amount” (as the case may be, although prophylaxis may be considered therapy), this being sufficient to show benefit to the individual. Typically, this will be to cause a therapeutically useful activity providing benefit to the individual. The actual amount of the compounds administered, and rate and time-course of administration, will depend on the nature and severity of the condition being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980 or Remington's Pharmaceutical Sciences, 19th edition, Mack Publishing Company, Easton, Pa., 1995; and Handbook of Pharmaceutical Excipients, 2nd edition, 1994. By way of example, and the compositions are preferably administered to patients in dosages of between about 0.01 and 100 mg of active compound per kg of body weight, and more preferably between about 0.5 and 10 mg/kg of body weight.

Experimental

Chalcones were prepared by the base catalysed condensation of an aldehyde and acetophenone. Those bearing a group at the alpha position were prepared by the Knoevenagel-like condensation of the appropriately substituted acetophenone and benzaldehyde.

Compounds disclosed here which have an amine functionality represent an important addition to the range of compounds which demonstrate significant activity. The amine functional groups allow the formation of salts which would enable the solubility properties of the compound to be altered, as well as influence the activity of the compound.

Chalcone structures bearing an alpha-alkoxy group are particularly active compounds.

Fluorinated versions of the chalcone structures are also active. Indeed, compounds with a fluorine at the 3 position on the B-ring demonstrate significant activity and DR5 is the most active fluorinated analogue.

Phosphate derivatives of the present invention also represent potent cytotoxins with enhanced solubility properties. Compounds SD174a and SD174b are potently active.

Indanones were prepared by trifluoroacetic acid catalysed ring closure of chalcones. These provided conformationally restricted chalcone analogues. Indanols were prepared by reduction of the indanones. Further reduction removed the oxygen functionalities altogether and related compounds were synthesised.

The compounds of the invention including quinone rings can be prepared using literature techniques from a monophenol by treatment with Fremy's salt to provide the quinone or from methoxyaryl, hydroxyaryl or aniline starting materials.

The synthesis of Boc-ester derivatives is disclosed in WO 02/50007.

The synthesis of compounds (e.g) of formula I in which the R₄ substituent comprises an amine or amide functional group such as —CH₂NH—R, where R is alkyl or —(C═O)—R, can be carried out starting from a parent ester. Reaction with BH₃ gives a —CH₂OH group that can be reacted under Mitsunobu conditions to give —CH₂-Phthalimide. This can then be alkylated or acylated using standard procedures.

For synthesizing —CH₂C═O compounds, standard techniques can be employed to convert an ester to CH₂OH (as above) then to CH₂Cl then to CH₂CN then to CH₂COOH. The acid can then be transformed into CH₂(C═O)—NHR and CH₂—(C═O)-alkyl or aryl groups.

The most active chalcone structures give the most active indanone compounds. Reduced forms of the indanones are less active than the parent ketone compounds. Interestingly, the highly reduced indanones are more active than the indanols.

Compounds based on the aurone structure were prepared as conformationally restricted analogues of the chalcones. They were prepared from the appropriate benzofuranone. Both DR27 and DR28 have significant activity, with IC₅₀ values in the cytotoxicity tests of 50 nM and 110 nM respectively.

The compounds disclosed here have been prepared and tested as racemic mixtures. It is expected that the pure enantiomers are likely to posses altered activity. The compounds of the invention will bind to proteins in the course of their action and therefore the chirality of the compound is likely to be important in determining their effectiveness.

Synthesis

Representative experimental details are presented here, together with analytical results for the exemplified compounds.

General Methods

Protocol E

To a stirring solution of substituted acetophenone and substituted benzaldehyde in alcohol was added a quantity of an aqueous solution of sodium hydroxide (50% w/v) and the mixture stirred at room temperature under argon overnight. The mixture was diluted with dichloromethane (50 cm³) and acidified to pH 1 with an aqueous solution of hydrochloric acid (50 cm³, 1 N). The separated aqueous layer was extracted further with dichloromethane (2×20 cm³) and the combined organic fractions dried over anhydrous magnesium sulphate, filtered and evaporated in vacuo. The residue was purified by column chromatography or recrystallisation.

Protocol F

The method adopted was similar to that of Giordano and co-workers (Giordano 1982). To a stirring solution of substituted phenacyl bromide in alcohol was added silver carbonate and boron trifluoride etherate. The solution was stirred at room temperature under argon for 2 days, filtered, diluted with dichloromethane (100 cm³), washed with water (50 cm³) and the organic fraction dried over anhydrous magnesium sulfate, filtered and evaporated in vacuo. The crude residue was purified by column chromatography.

Protocol G

The method adopted was that of Varma and co-workers (Varma 1992). To a stirring solution of substituted benzophenone and substituted benzaldehyde in dichloromethane was added neutral alumina and the mixture stirred at room temperature under argon for 1-3 days. The mixture was filtered, diluted with dichloromethane (20 cm 3), washed with distilled water (10 cm³), dried over anhydrous magnesium sulfate, filtered and evaporated in vacuo. The crude residue was purified by either column chromatography or recrystallisation.

Protocol H

The method adopted was that of Wheeler and co-workers (Fitzgerald 1955). A solution of aurone and potassium cyanide in ethanol/dichloromethane was heated at reflux under argon for 12 h. The mixture was poured into water (15 cm³) and extracted with dichloromethane (3×10 cm³), the combined organic fractions dried over anhydrous magnesium sulfate, filtered and evaporated in vacuo. The crude residue was purified by column chromatography.

3-(3″-Hydroxy-4″-methoxy-phenyl) 3′,4′,5′-trimethoxy-1-indanone (DM13).

General procedure: A red solution of chalcone (3.05 mmol) in TFA (100 mL) was heated under reflux for 6 hours. The TFA was then distilled and the residue was extracted with chloroform (50-100 mL). The organic extract was treated with NaHCO₃ solution (1M, 2×50 mL) and water (100 mL). The organic layer was dried over MgSO₄, and the solvent was evaporated in vacuo, leaving the product as a yellow-brown solid.

The indanone DM13 was obtained by the general procedure using 1-(3″-hydroxy-4″-methoxyphenyl)-3-(3′,4′,5′-trimethoxyphenyl)-1-propen-3-one (1 g, 2.9 mmol) in TFA (100 mL), giving a brown solid (910 mg, 91%).

m.p. 110-112° C.; δ_{H} (300 MHz, CDCl₃) 2.60 (1H, dd, J 2.26 Hz, 19.2 Hz, H2a), 3.2 (1H, dd, J 7.9 Hz; 19.2 Hz, H2b), 3.45 (3H, s, OCH₃), 3.87 (3H, s, OCH₃), 3.92 (3H, s, OCH₃), 3.93 (3H, s, OCH₃), 4.5 (1H, dd, J 2.26 Hz, 7.9 Hz, H3), 5.56 (1H, s, OH) 6.6 (1H, d, J 1.88 Hz, H2″), 6.65 (1H, dd, J 1.88 Hz, 7.91 Hz, H6″), 6.82 (1H, d, J 7.91 Hz, H5″), 7.09 (1H, s, H6′); δ_{C} (75 MHz, CDCl₃) 41.4 (CH, C3), 47.7 (CH2, C2), 56.3, 56.6, 60.5, 61.3 (CH₃), 100.7 (CH, C6′), 111.0 (CH, C2″), 113.7 (CH, C6″), 119.1 (CH, C5″), 132.6, 138.1, 145.0, 145.6, 146.1, 149.2, 150.8, 155.2, 205.8 (C); vₘₐₓ (KBr disc) 3230 (OH), 1700 (C═O), 1600 (C═C), 1510, 1470, 1350, 1275, 1220 (C—O), 1140, 1100, 1030 cm⁻¹; m/z (FAB) 345 [(M+H)⁺, 100%]; (Found: C, 66.4; H, 6.0. C₁₉H₂₀O₆ requires C, 66.2; H, 5.8%).

(E)-3-(4″-Methoxy-3″-nitrophenyl)-1-(3′,4′,5′-trimethoxyphenyl)-2-propen-1-one (MW47).

A mixture of 3,4,5-trimethoxyacetophenone (2.0 g, 9.5 mmol), 4-methoxy-3-nitrobenzaldehyde(1.7 g, 9.5 mmol) and sodium hydroxide solution (0.4 g in 1 cm³ of water) in methanol (10 cm³) was stirred at room temperature overnight. The subsequent mixture was acidified with 1N hydrochloric acid (20 cm³) and extracted with chloroform (50 cm³). The organic layer was separated, dried over MgSO₄ and concentrated in vacuo. Purification by recrystallisation from ethyl acetate afforded the chalcone MW47 as a pale orange solid (2.2 g, 61%).

m.p. 143-145° C.; δ_{H} (300 MHz, CDCl₃) 3.95 (3H, s, OCH₃), 3.97 (6H, s, OCH₃), 4.02 (3H, s, OCH₃), 7.14 (1H, d, J 8.7 Hz, H-5″), 7.29 (2H, s, H-2′, H-6′), 7.45 (1H, d, J 15.5 Hz, H-2), 7.75 (1H, d, J 15.5 Hz, H-3), 7.79 (1H, dd, J 8.7 and 2.3 Hz, H-6″), 8.17 (1H, d, J 2.3 Hz, H-2″); δ_{C} (75 MHz, CDCl₃) 56.8 (OCH₃), 57.2 (OCH₃), 61.4 (OCH₃), 106.5 (CH), 114.2 (CH), 122.3 (CH), 125.1 (CH), 128.0 (C), 133.5 (C), 134.9 (CH), 140.3 (C), 142.0 (CH), 143.2 (C), 153.6 (C), 154.5 (C), 188.8 (C═O); v_{max.} (KBr) 1005 (s), 1030 (w), 1070 (w), 1090 (w), 1130 (s), 1160 (m), 1180 (w), 1215 (m), 1235-1250 (v), 1280 (s), 1310 (w), 1320 (w), 1350 (s), 1420 (s), 1460-1475 (v), 1505 (s), 1530 (s), 1565-1580 (v), 1600 (s), 1620 (m), 1655 (s), 2840 (m), 2930 (w), 2960 (m), 3000 (m), 3040-3070 (v) cm⁻¹ 1; m/z (FAB) 374 ([M+H]⁺, 100%). Found C, 61.3; H, 5.1; N, 3.9%. C₁₉H₁₉NO₇ requires C, 61.1; H, 5.1; N, 3.8%.

(E)-3-(3″-Amino-4″-methoxyphenyl)-1-(3′,4′,5′-trimethoxyphenyl)-2-propen-1-one (MW65).

A mixture of (E)-3-(4″-methoxy-3″-nitrophenyl)-1-(3′,4′,5′-trimethoxyphenyl)-2-propen-1-one (MW47)(1.00 g, 2.7 mmol), tin(II) chloride dihydrate (3.02 g, 13.4 mmol) and concentrated hydrochloric acid (10 drops) in 1:1 ethanol:ethyl acetate (20 cm³) was stirred and heated to reflux for 2 days. The cooled mixture was diluted with ethyl acetate (30 cm³) and washed with saturated sodium hydrogen carbonate solution (20 cm³) followed by brine (20 cm³). The organic layer was separated, dried over MgSO₄ and concentrated in vacuo. Purification by column chromatography (SiO₂, chloroform:ethyl acetate 4:1) afforded the chalcone MW65 as an orange yellow solid (0.29 g, 32%).

m.p. 90-91° C.; R_{f} 0.49 (SiO₂, chloroform:ethyl acetate 4:1); δ_{H} (300 MHz, CDCl₃) 3.92 (3H, s, OCH₃), 3.95 (3H, s, OCH₃), 3.96 (6H, s, OCH₃), 6.82 (1H, d, J 7.9 Hz, H-5″), 7.04 (1H, s, H-2″), 7.07 (1H, d, J 7.9 Hz, H-6″), 7.28 (2H, s, H-2′, H-6′), 7.31 (1H, d, J 15.5 Hz, H-2), 7.73 (1H, d, J 15.5 Hz, H-3); δ_{C} (75 MHz, CDCl₃) 56.0 (OCH₃), 56.8 (OCH₃), 61.4 (OCH₃), 106.4 (CH), 110.6 (CH), 113.7 (CH), 119.7 (CH), 121.4 (CH), 128.4 (C), 134.4 (C), 136.9 (C), 142.6 (C), 145.7 (CH), 150.1 (C) 153.5 (C), 189.9 (C═O); v_{max.} (KBr) 1000 (m), 1030 (m), 1070 (w), 1130 (s), 1160 (s), 1090 (w0, 1230-1240 (v), 1270 (m), 1300 (w0, 1315 (m), 1335-1355 (v), 1420 (s), 1435-1470 (v), 1510-1520 (v), 1560-1580 (v), 1655 (s), 2840 (m), 2900-2980 (v), 3000 (w), 3370 (s), 3460 (m) cm⁻¹; m/z (EI) 343 ([M]^{+.}, 100%). Found C, 66.5; H, 6.2; N, 4.1%. C₁₉H₂₁NO₅ requires C, 66.5; H, 6.2; N, 4.1%.

4,5,6-Trimethoxy-3-(4′-methoxy-3′-nitrophenyl)-1-indanone (MW73).

A red solution of (E)-3-(4″-methoxy-3″-nitrophenyl)-1-(3′,4′,5′-trimethoxyphenyl)-2-propen-1-one (MW47)(1.00 g, 2.68 mmol) in TFA (1.7 cm³) was stirred and heated to reflux overnight. To the cooled solution was added the ice-cold water (20 cm³). The mixture was extracted with ethyl acetate (50 cm³). The organic layer was separated, dried over MgSO₄ and concentrated in vacuo. Purification by column chromatography (SiO₂, hexane:ethyl acetate 2:1) and recrystallisation from 2:1 hexane:ethyl acetate afforded the indanone MW73 a pale yellow solid (0.76 g, 76%).

m.p. 134-136° C.; R_{f} 0.21 (SiO₂, hexane:ethyl acetate 2:1); δ_{H} (300 MHz, CDCl₃) 2.57 (1H, dd, J 19.2 and 2.6 Hz, H-2), 3.23 (1H, dd, J 19.2 and 8.3 Hz, H-2), 3.52 (3H, s, OCH₃), 3.92 (3H, s, OCH₃), 3.94 (3H, s, OCH₃), 3.95 (3H, s, OCH₃), 4.60 (1H, dd, J 8.3 and 2.6 Hz, H-3), 7.02 (1H, d, J 8.7 Hz, H-5′), 7.10 (1H, s, H-7), 7.27 (1H, dd, J 8.7 and 2.3 Hz, H-6′), 7.65 (1H, d, J 2.3 Hz, H-2′); δ_{c} (75 MHz, CDCl₃) 40.7 (CH), 47.1 (CH₂), 56.7 (OCH₃), 57.0 (OCH₃), 60.6 (OCH₃), 61.3 (OCH₃), 100.8 (CH), 114.2 (CH), 124.8 (CH), 132.5 (C), 133.0 (CH), 137.1 (C), 139.9 (C), 143.2 (C), 149.0 (C), 150.6 (C), 152.0 (C), 155.8 (C), 204.5 (C═O); v_{max.} (KBr) 1010 (m), 1030 (w), 1040 (w), 1100 (s), 1135 (s), 1160 (w), 1200 (m), 1215 (m), 1230 (w), 1260 (m), 1280 (s), 1320 (m), 1330 (m), 1350 (s), 1425 (m), 1450-1485 (v), 1520-1540 (b), 1570 (m), 1600 (m), 1625 (m), 1700-1720 (b), 2370 (w), 2840 (w), 2900-2970 (v), 3000 (w) cm⁻¹; m/z (FAB) 374 ([M]+, 40%), 43 (100%).

Found C, 61.1; H, 5.3; N, 3.7%. C₁₉H₁₉NO₇ requires C, 61.1; H, 5.1; N, 3.8%.

3-(3′-Amino-4′-methoxyphenyl)-4,5,6-trimethoxy-1-indanone (MW74).

To a stirring activated suspension of 10% Pd/C (1 spatula) in methanol (5 cm³) was injected a solution of 4,5,6-trimethoxy-3-(4′-methoxy-3′-nitrophenyl)-1-indanone (MW73)(0.20 g, 0.54 mmol) in methanol (20 cm³). The mixture was stirred at room temperature under a hydrogen atmosphere for 90 min., filtered through celite and evaporated in vacuo to give the indanone MW74 as an orange liquid (0.18 g, 97%).

δ_{H} (300 MHz, CDCl₃) 2.60 (1H, dd, J 19.2 and 2.6 Hz, H-2), 3.15 (1H, dd, J 19.2 and 7.9 Hz, H-2) 3.42 (3H, s, OCH₃), 3.82 (3H, s, OCH₃), 3.91 (3H, s, OCH₃), 3.92 (3H, s, OCH₃), 4.47 (1H, dd, J 7.9 and 2.6 Hz, H-3), 6.42 (1H, d, J 2.3 Hz, H-2′), 6.50 (1H, dd, J 8.3 and 2.3 Hz, H-6′), 6.70 (1H, d, J 7.9 Hz, HH-5′), 7.09 (1H, s, H-7); δ_{C} (75 MHz, CDCl₃) 41.5 (CH), 47.8 (CH₂), 55.9 (OCH₃), 56.6 (OCH₃), 60.6 (OCH3), 61.2 (OCH₃), 100.6 (CH), 110.7 (CH), 114.0 (CH), 117.6 (CH), 132.5 (C), 136.6 (C), 137.5 (C), 145.4 (C), 146.5 (C), 149.2 (C), 150.8 (C), 155.1 (C), 206.1 (C═O); v_{max.} (KBr) 1005 (w), 1030 (s), 1100 (s), 1130 (s), 1170 (m), 1210-1240 (v), 1260 (w), 1315 (s), 1345 (s), 1420-1430 (v), 1450-1470 (v), 1520 (s), 1600 (s), 1620 (m), 1700-1720 (b), 2840 (m), 2910-2980 (v), 3000 (w), 3380 (s), 3440-3480 (b) cm⁻¹; m/z (FAB) 343 ([M]^{+.}, 100%). Found C, 66.2; H, 6.1; N, 3.8%. C₁₉H₂₁NO₅ requires C, 66.5; H, 6.2; N, 4.1%.

(E)-3-(3″-Hydroxy-4″-methoxyphenyl)-1-(2′,3′,4′-trimethoxyphenyl)-2-propen-1-one (DRS).

The chalcone DR8 was obtained following the general protocol E using 2,3,4-trimethoxyacetophenone (0.50 g, 2.38 mmol), 3-hydroxy-4-methoxybenzaldehyde (0.36 g, 2.38 mmol) and sodium hydroxide (0.5 cm³, 50% w/v) in methanol (10 cm³), with recrystallisation from methanol affording DR8 as a yellow solid (0.38 g, 1.56 mmol, 66%).

m.p. 85-86° C.; δ_{H} (300 MHz, CDCl₃) 3.90 (12H, s, OMe), 5.73 (1H, s, OH), 6.74 (1H, d, J 8.8 Hz, H-5′), 6.86 (1H, d, J 8.1 Hz, H-5″), 7.10 (1H, dd, J 8.1 and 2.1 Hz, H-6″) , 7.26 (1H, d, J 2.1 Hz, H-2″) , 7.36 (1H, d, J 15.8 Hz, H-2), 7.38 (1H, d, J 8.8 Hz, H-6′), 8.61 (1H, d, J 15.8 Hz, H-3); δ_{C} (75 MHz, CDCl₃) 56.4 (CH₃), 56.5 (CH₃), 61.4 (CH₃), 62.4 (CH₃), 107.7 (CH), 111.0 (CH), 113.5 (CH), 122.8 (CH), 125.3 (CH), 126.1 (CH), 127.4 (C), 129.2 (C), 142.6 (C), 143.5 (CH), 146.3 (C), 149.0 (C), 154.1 (C), 157.3 (C), 191.3 (C); vₘₐₓ (KBr disc) 3400, 1600, 1510, 1460, 1270, 1100 cm⁻¹; m/z (FAB) 244 [M+, 65%]; (Found C, 66.2; H, 6.2. C₁₉H₂₀O₆ requires C, 66.3; H, 5.9%).

(Z)-3-(3″-Hydroxy-4″-methoxyphenyl)-2-methoxy-1-(3′,4′,5′-timethoxyphenyl)-2-propen-1-one (DR13).

To a stirring solution of 2-methoxy-1-(3′,4′,5′-trimethoxyphenyl)ethan-1-one (1.00 g, 4.2 mmol) and 3-hydroxy-4-methoxybenzaldehyde (0.64 g, 4.2 mmol) in methanol (15 cm³) was added sodium hydroxide (6.00 g, 150.0 mmol) to give a solution concentration of 10 N. The mixture was stirred at room temperature under argon overnight, diluted with water (50 cm³), acidified to pH 1 with concentrated hydrochloric and extracted with chloroform (2×25 cm³). The combined organic fractions were dried over anhydrous magnesium sulfate, filtered and evaporated in vacuo. Purification by column chromatography (SiO₂, hexane:ethyl acetate 2:1) afforded DR13 as a yellow solid (0.48 g, 1.28 mmol, 31%).

m.p. 120-122° C.; δ_{H} (300 MHz, CDCl₃) 3.77 (3H, s, OMe), 3.91 (6H, s, OMe), 3.93 (3H, s, OMe), 3.94 (3H, s, OMe), 5.62 (1H, s, OH), 6.85 (1H, d, J 8.6 Hz, H-5″) , 6.46 (1H, s, H-3), 7.18 (2H, s, H-2′, H-6′), 7.21 (1H, dd, J 8.6 and 2.1 Hz, H-6″) , 7.53 (1H, d, J 2.1 Hz, H-2″) ; δ_{c} (75 MHz, CDCl₃) 56.3 (CH₃), 56.7 (CH₃), 58.9 (CH₃), 61.3 (CH₃), 107.5 (CH), 110.8 (CH), 116.3 (CH), 123.7 (CH), 124.6 (CH), 127.8 (C), 133.2 (C), 142.6 (C), 145.8 (C), 147.7 (C), 152.5 (C), 153.4 (C), 192.0 (C); vₘₐₓ (KBr disc) 3420, 2950, 1650, 1620, 1590, 1500, 1420, 1340, 1130 cm⁻¹; m/z (FAB) 374 [M⁺, 100%]), 195 (100); (Found C, 64.5; H, 6.2. C₂₀H₂₂O₇ requires C, 64.2; H, 5.9%).

2-Methoxy-1-(3,4,5-trimethoxy-phenyl-ethanone.

The ketone was obtained following protocol F using 2-bromo-1-(3′,4′,5′-trimethoxyphenyl)ethan-1-one (4.18 g, 14.5 mmol), silver(I) carbonate (5.00 g, 18.2 mmol) and boron trifluoride etherate (2.10 cm³, 16.7 mmol) in methanol (40 cm³). Purification by column chromatograghy (SiO₂, hexane:ethyl acetate 2:1) afforded the ketone as a white solid (2.57 g, 10.7 mmol, 74%).

m.p. 54-55° C. (Pratt et al 1925 reported m.p. 54° C.); δ_{H} (300 MHz, CDCl₃) 3.51 (3H, s, OMe), 3.93 (9H, s, OMe), 4.68 (2H, s, CH₂), 7.20 (2H, s, H-2′, H-6′); δ_{c} 56.4 (CH₃), 59.5 (CH₃), 61.0 (CH₃), 72.3 (CH₂), 102.0 (CH), 130.1 (C), 143.0 (C), 153.2 (C), 195.0 (C); vₘₐₓ (KBr disc) 3010, 2950, 1690, 1590, 1420, 1340, 1140 cm⁻¹; m/z (FAB) 241 [MH⁺, 100%], 195 (90); Found C, 60.1; H, 6.8. C₁₂H₁₆O₅ requires C, 60.0; H, 6.7%).

2-Bromo-1-(3′,4′,5′-trimethoxyphenyl)ethan-1-one.

To a stirring solution of 3,4,5-trimethoxyacetophenone (10.00 g, 47.6 mmol) in dry diethyl ether (450 cm³) at 0° C. under argon was added bromine (2.70 cm³, 52.3 mmol) in dry ether (250 cm³). On completion of addition the flask was irradiated with a 125 W light source for 1 h. The mixture was washed with an aqueous solution (saturated) of sodium metabisulfite (2×200 cm³) and the organic fraction dried over anhydrous magnesium sulfate, filtered and evaporated in vacuo. Recrystallisation from diethyl ether afforded 2-bromo-1-(3′,4′,5′-trimethoxyphenyl)ethan-1-one as a white solid (11.60 g, 40.3 mmol, 85%).

m.p. 64-66° C. (Horton et al. 1954 reported m.p. 63-67° C.); δ_{H} (300 MHz, CDCl₃) 3.94 (9H, s, OMe), 4.41 (2H, s, CH₂), 7.22 (2H, s, H-2′, H-6′); δ_{C} (75 MHz, CDCl₃) 30.6 (CH₂), 56.4 (CH₃), 61.1 (CH₃), 106.6 (CH), 129.0 (C), 143.4 (C), 153.2 (C), 190.3 (C); vₘₐₓ (KBr disc) 2950, 2850, 1690, 1590, 1410, 1340, 1130 cm⁻¹; m/z (FAB) 291 [MH⁺, ⁸¹Br, 40%], 289 [MH⁺, ⁷⁹ Br, 45%], 195 (100); Found C, 46.0; H, 4.5. C₁₁H₁₃O₄Br requires C, 45.7; H, 4.5%).

(Z)-3-(3″-Fluoro-4″-methoxyphenyl)-2-methoxy-1-(3′,4′,5′-trimethoxyphenyl)-2-propen-1-one (DR14).

The chalcone DR14 was obtained following protocol E using 2-methoxy-1-(3,4,5-trimethoxyphenyl)-ethanone (0.30 g, 1.25 mmol), 3-fluoro-4-methoxybenzaldehyde (0.19 g, 1.25 mmol) and sodium hydroxide (0.50 cm³, 3 N) in methanol (4 cm³), with purification by column chromatography (SiO₂, hexane:ethyl acetate 2:1) affording DR14 as a yellow solid (0.29 g, 0.77 mmol, 62%).

m.p. 110-112° C.; δ_{H} (400 MHz, CDCl₃) 3.78 (3H, s, OMe), 3.92 (3H, s, OMe), 3.93 (6H, s, OMe), 3.95 (3H, s, OMe), 6.41 (1H, s, H-3), 6.95 (1H, t, J 8.6 Hz, H-5″) , 7.19 (2H, s, H-2′, H-6′), 7.37 (1H, d, J 8.6 Hz, H-6″) , 7.74 (1H, dd, J 13.0 and 2.0 Hz, H-2″) ; δ_{C} (100 MHz, CDCl₃) δ6.6 (CH₃), 56.8 (CH₃), 59.0 (CH₃), 61.4 (CH₃), 107.4 (CH), 113.2 (CH, d, J 3.0 Hz), 117.6 (CH, d, J 15.0 Hz), 122.8 (CH, d, J 3.0 Hz), 127.3 (CH, d, J 6.0 Hz), 127.5 (C, d, J 6.0 Hz), 132.9 (C), 142.7 (C), 148.6 (C, d, J 15.0 Hz), 152.4 (C, d, J 245.0 Hz), 152.9 (C), 153.4 (C), 191.7 (C); δ_{F} (200 MHz, CDCl₃); vₘₐₓ (KBr disc) 1660, 1610, 1580, 1510, 1470, 1420, 1330, 1270, 1140 cm⁻¹; m/z (FAB) 377 [MH⁺, 100%]; (Found C, 63.8; H, 5.8. C₂₀H₂₁O₆F requires C, 63.8; H, 5.6%).

(Z)-3-(3″,5″-Difluoro-4″-methoxyphenyl)-2-methoxy-1-(3′,4′,5′-trimethoxyphenyl)-2-propen-1-one (DR16).

The chalcone DR16 was obtained following protocol E using 2-methoxy-1-(3,4,5-trimethoxyphenyl)-ethanone (0.30 g, 1.25 mmol), 3,5-difluoro-4-methoxybenzaldehyde (0.22 g, 1.25 mmol) and sodium hydroxide (0.50 cm³, 3 N) in methanol (4 cm³), with purification by column chromatography (SiO₂, hexane:ethyl acetate 3:1) affording DR16 as a yellow solid (0.37 g, 0.94 mmol, 75%).

m.p. 124-126° C.; δ_{H} (400 MHz, CDCl₃) 3.79 (3H, s, OMe), 3.92 (6H, s, OMe), 3.96 (3H, s, OMe), 4.04 (3H, s, OMe), 6.23 (1H, S, H-3), 7.20 (2H, s, H-2′, H-6′), 7.34 (2H, d, J 9.9 Hz, H-2″, H-6″) ; δ_{C} (100 MHz, CDCl₃) 56.8 (CH₃), 59.0 (CH₃), 61.4 (CH₃), 62.3 (CH₃), 107.4 (CH), 114.0 (CH, dd, J 13.0 and 3.0 Hz), 119.8 (CH, t, J 3.0 Hz), 129.0 (C, t, J 7.0 Hz), 132.3 (C), 136.9 (C, t, J 13.0 Hz), 143.1 (C), 153.4 (C), 154.1 (C), 155.6 (C, dd, J 244.0 and 7.0 Hz), 191.3 (C); δ₈F (200 MHz, CDCl₃); vₘₐₓ (KBr disc) 1640, 1580, 1500, 1450, 1330, 1240, 1130 cm⁻¹; m/z (FAB) 395 [MH⁺, 100%]; (Found C, 61.2; H, 5.4. C₂₀H₂₀O₆F₂ requires C, 60.9; H, 5.1%).

(Z)-3-(3″-Fluoro-4″-methoxyphenyl)-2-ethoxy-1-(3′,4′,5′-trimethoxyphenyl)-2-propen-1-one (DR17).

The chalcone DR17 was obtained following protocol E using 2-ethoxy-1-(3′,4′,5′-trimethoxyphenyl)-1-ethanone (0.30 g, 1.18 mmol), 3-fluoro-4-methoxybenzaldehyde (0.18 g, 1.18 mmol) and sodium hydroxide (1.00 cm³, 3 N) in ethanol (4 cm³), with purification by column chromatography (SiO₂, hexane:ethyl acetate 5:2) affording DR17 as a yellow solid (0.25 g, 0.64 mmol, 54%).

m.p. 89-90° C.; δ_{H} (300 MHz, CDCl₃) 1.38 (3H, t, J 7.0 Hz, H-5), 3.92 (6H, s, OMe), 3.93 (3H, s, OMe), 3.95 (3H, s, OMe), 3.99 (2H, q, J 7.0 Hz, H-4), 6.43 (1H, s, H-3), 6.95 (1H, t, J 8.8 Hz, H-5″) , 7.22 (2H, s, H-2′, H-6′), 7.40 (1H, d, J 8.8 Hz, H-6″) , 7.80 (1H, dd, J 13.2 and 2.2 Hz, H-2″) ; δ_{C} (75 MHz, CDCl₃) 16.0 (CH₃), 56.6 (CH₃), 56.7 (CH₃), 61.4 (CH₃), 67.4 (CH₂), 107.4 (CH), 113.3 (CH, d, J 3.0 Hz), 117.6 (CH, d, J 15.0 Hz), 122.6 (CH, d, J 3.0 Hz), 127.2 (CH, d, J 6.0 Hz), 127.7 (C, d, J 6.0 Hz), 132.7 (C), 142.8 (C), 148.5 (C, d, J 15.0 Hz), 152.1 (C), 152.4 (C, d, J 245.0 Hz), 153.3 (C), 191.9 (C); δ_{F} (200 MHz, CDCl₃); vₘₐₓ (KBr disc) 1580, 1520, 1460, 1420, 1330, 1280, 1130 cm⁻¹; m/z (FAB) 391 [MH⁺, 90%]; (Found C, 64.8; H, 5.7. C₂₁H₂₃O₆F requires C, 64.6; H, 5.9%).

2-Ethoxy-1-(3′,4′,5′-trimethoxyphenyl)-1-ethanone.

The ketone was obtained following protocol F using 2-bromo-1-(3′,4′,5′-trimethoxyphenyl)-1-ethanone (3.00 g, 10.4 mmol), silver(I) carbonate (3.58 g, 13.0 mmol) and boron trifluoride etherate (1.50 cm³, 12.0 mmol) in ethanol (60 cm³). Purification by column chromatography (SiO₂, hexane:ethyl acetate 3:1) afforded the ketone as a pale yellow oil (2.42 g, 9.5 mmol, 91%).

δ_{H} (400 MHz, CDCl₃) 1.28 (3H, t, J 7.0 Hz, H-4), 3.63 (2H, q, J 7.0 Hz, H-3), 3.90 (9H, s, OMe), 4.68 (2H, s, CH₂), 7.22 (2H, s, H-2′, H-6′); δ_{C} (100 MHz, CDCl₃) 15.5 (CH₃), 56.7 (CH₃), 61.3 (CH₃), 67.6 (CH₂), 74.1 (CH₂), 105.9 (CH), 130.5 (C), 143.3 (C), 153.5 (C), 195.8 (C); vₘₐₓ (KBr disc) 1700, 1590, 1510, 1460, 1420, 1330, 1240, 1130 cm⁻¹; m/z (FAB) 255 [MH⁺, 100%].

(Z)-3-(3″-Fluoro-4″-methoxyphenyl)-2-propoxy-1-(3′,4′,5′-trimethoxyphenyl)-2-propen-1-one (DR20).

The chalcone DR20 was obtained following protocol E using 2-propoxy-1-(3′,4′,5′-trimethoxyphenyl)-1-ethanone (0.32 g, 1.19 mmol), 3-fluoro-4-methoxybenzaldehyde (0.18 g, 1.19 mmol) and sodium hydroxide (1.00 cm³, 3 N) in propanol (4 cm³), with purification by column chromatography (SiO₂, hexane:ethyl acetate 2:1) affording DR20 as a yellow solid (0.29 g, 0.72 mmol, 61%).

m.p. 82-83° C.; δ_{H} (400 MHz, CDCl₃) 1.00 (3H, t, J 7.2 Hz, H-6), 1.77 (2H, sextet, J 7.2 Hz, H-5), 3.87 (2H, t, J 7.2 Hz, H-4), 3.92 (6H, s, OMe), 3.93 (3H, s, OMe), 3.95 (3H, s, OMe), 6.38 (1H, s, H-3), 6.95 (1H, t, J 8.5 Hz, H-5″) , 7.23 (2H, s, H-2′, H-6′), 7.39 (1H, d, J 8.5 Hz, H-6″) , 7.79 (1H, dd, J 13.2 and 2.3 Hz, H-2″) ; δ_{C} (100 MHz, CDCl₃) 10.8 (CH₃), 23.8 (CH₂), 56.6 (CH₃), 56.7 (CH₃), 61.4 (CH₃), 73.3 (CH₂), 107.7 (CH), 113.3 (CH, d, J 3.0 Hz), 117.6 (CH, d, J 15.0 Hz), 121.9 (CH, d, J 3.0 Hz), 127.2 (CH, d, J 6.0 Hz), 127.8 (C, d, J 6.0 Hz), 132.7 (C), 142.8 (C), 148.4 (C, d, J 15.0 Hz), 152.3 (C, d, J 245.0 Hz), 152.4 (C), 153.3 (C), 191.9 (C); δ_{F} (200 MHz, CDCl₃); vₘₐₓ (KBr disc) 1650, 1580, 1520, 1420, 1240, 1130 cm⁻¹; m/z (FAB) 405 [MH⁺, 60%]; (Found C, 65.6; H, 6.0. C₂₂H₂₅O₆F requires C, 65.3; H, 6.2%).

2-Propoxy-1-(3′,4′,5′-trimethoxyphenyl)-1-ethanone.

The ketone was obtained following protocol F using 2-bromo-1-(3′,4′,5′-trimethoxyphenyl)-1-ethanone (4.00 g, 13.8 mmol), silver(I) carbonate (4.76 g, 17.3 mmol) and boron trifluoride etherate (2.00 cm³, 15.9 mmol) in propanol (60 cm³). Purification by column chromatography (SiO₂, hexane:ethyl acetate 2:1) afforded the ketone as a colourless oil (2.30 g, 8.6 mmol, 62%).

δ_{H} (400 MHz, CDCl₃) 0.95 (3H, t, J 7.2 Hz, H-5), 1.68 (2H, sextet, J 7.2 Hz, H-4), 3.53 (2H, t, J 7.2 Hz, H-3), 3.91 (9H, s, OMe), 4.68 (2H, s, CH₂), 7.25 (2H, s, H-2′, H-6′); δ_{C} (100 MHz, CDCl₃) 10.9 (CH₃), 23.3 (CH₂), 56.7 (CH₃), 61.4 (CH₃), 73.9 (CH₂), 74.4 (CH₂), 106.0 (CH), 130.6 (C), 143.3 (C), 153.5 (C), 196.0 (C); vₘₐₓ (KBr disc) 1700, 1590, 1500, 1460, 1420, 1240, 1130 cm⁻¹; m/z (FAB) 269 [MH⁺, 70%]; (Found C, 62.9; H, 7.3. C₁₄H₂₀O₅ requires C, 62.7; H, 7.5%).

2-[(Z)-(3′-Hydroxy-4′-methoxyphenyl)methylidene]-5,6,7-trimethoxy-1-benzofuran-3-one (DR27).

The aurone DR27 was obtained following protocol G using 5,6,7-trimethoxy-1-benzofuran-3(2H)-one (0.21 g, 0.94 mmol), 3-hydroxy-4-methoxybenzaldehyde (0.14 g, 0.94 mmol) and neutral alumina (3.00 g) in dichloromethane (2 cm³) stirring for 3 days, with purification by column chromatography (SiO₂, hexane:ethyl acetate 1:1) affording DR27 as an orange solid (0.16 g, 0.45 mmol, 48%).

m.p. 192-193° C.; δ_{H} (300 MHz, CDCl₃) 3.89 (3H, s, OMe), 3.97 (3H, s, OMe), 4.04 (3H, s, OMe), 4.23 (3H, s, OMe), 5.70 (1H, s, OH), 6.82 (1H, s, H-8), 6.94 (1H, d, J 8.4 Hz, H-5′), 7.00 (1H, s, H-4), 7.39 (1H, dd, J 8.4 and 1.9 Hz, H-6′), 7.59 (1H, d, J 1.9 Hz, H-2′); δ_{C} (75 MHz, CDCl₃) 56.4 (CH₃), 56.8 (CH₃), 61.6 (CH₃), 62.0 (CH₃), 99.7 (CH), 111.1 (CH), 113.6 (CH), 117.2 (CH), 125.4 (CH), 126.2 (C), 139.3 (C), 146.2 (C), 146.7 (C), 148.6 (C), 149.3 (C), 150.9 (C), 154.2 (C), 184.1 (C); vₘₐₓ (KBr disc) 3250, 1690, 1640, 1590, 1500, 1350, 1290 cm⁻¹; m/z (FAB) 359 [MH⁺, 100%]; (Found C, 64.1; H, 5.0. C₁₉H₁₈O₇ requires C, 63.7; H, 5.1%).

5,6,7-Trimethoxy-1-benzofuran-3(2H)-one.

The method adopted was that of Mahajan and co-workers (Mahajan 1996). A solution of 2,3,4-trimethoxyphenoxyacetic acid (3.87 g, 16.0 mmol) in polyphosphoric acid (75 cm³) was heated at 80° C. under argon for 8 h. The mixture was poured into water (250 cm³) and extracted with dichloromethane (4×50 cm³), and the combined organic fractions dried over anhydrous magnesium sulfate and evaporated in vacuo. Purification by column chromatography (SiO₂, hexane:ethyl acetate 2:1) afforded 5,6,7-trimethoxy-1-benzofuran-3 (2H)-one as a pale brown solid (2.08 g, 9.3 mmol, 58%).

m.p. 81-83° C.; δ_{H} (400 MHz, CDCl₃) 3.83 (3H, s, OMe), 3.99 (3H, s, OMe), 4.02 (3H, s, OMe), 4.62 (2H, s, CH₂), 6.82 (2H, s, H-2, H-6); δ_{C} (100 MHz, CDCl₃) δ6.7 (CH₃), 61.5 (CH₃), 61.8 (CH₃), 75.5 (CH₂), 98.6 (CH), 116.2 (C), 139.5 (C), 150.0 (C), 150.5 (C), 163.3 (C), 199.2 (C); vₘₐₓ (KBr disc) 1690, 1610, 1480, 1260, 1110 cm⁻¹; m/z (FAB) 225 [MH⁺, 80%]; (Found C, 59.0; H, 5.4. C₁₁H₁₂O₅ requires C, 58.9; H, 5.4%).

2,3,4-Trimethoxyphenoxyacetic Acid.

The method adopted was similar to that of Abraham and co-workers (Abraham 1984). To a solution of 2,3,4-trimethoxyphenol (6.60 g, 35.9 mmol) in anhydrous dimethylformamide (100 cm³) was added sodium hydride (2.16 g, 89.8 mmol) and chloroacetic acid (3.39 g, 35.9 mmol) in anhydrous dimethylformamide (25 cm³). The mixture was stirred at room temperature under argon overnight, diluted with dichloromethane (200 cm³) and the organic fraction washed with water (100 cm³) and an aqueous solution of hydrochloric acid (400 cm³, 1 N). The separated aqueous layer was extracted further with dichloromethane (3×100 cm³) and the combined organic fractions dried over anhydrous magnesium sulfate, filtered and evaporated in vacuo. Purification by column chromatography (SiO₂, 3% methanol in chloroform) afforded 2,3,4-trimethoxyphenoxyacetic acid as a pale brown solid (6.99 g, 28.9 mmol, 81%).

m.p. 102-104° C.; δ_{H} (400 MHz, CDCl₃) 3.84 (3H, s, OMe), 3.91 (3H, s, OMe), 3.96 (3H, s, OMe), 4.66 (2H, s, CH₂), 6.59 (1H, d, J 9.4 Hz, H-5,), 6.67 (1H, d, J 9.4 Hz, H-6); δ_{C} (100 MHz, CDCl₃) 56.7 (CH₃), 61.6 (CH₃), 62.0 (CH₃), 68.8 (CH₂), 107.1 (CH), 111.6 (CH), 143.6 (C), 144.7 (C), 145.9 (C), 150.1 (C), 173.1 (C); vₘₐₓ (KBr disc) 3000, 1720, 1500, 1270, 1100 cm⁻¹; m/z (FAB) 242 [M⁺, 100%]; (Found C, 54.7; H, 5.8. C₁₁H₁₄O₆ requires C, 54.5; H, 5.8%).

The synthesis of compounds represented by formula (IV) will be known to those skilled in the art, but the synthesis of two compounds represented by formula (IV) is described here.

2-(3′-Hydroxy-4′-methoxyphenyl)-5,6,7-trimethoxy-4H-chromen-4-one (DR33).

The flavone DR33 was obtained following protocol H using DR23 (72 mg, 0.20 mmol) and potassium cyanide (130 mg, 2.00 mmol) in ethanol (3 cm³) and dichloromethane (2 cm³), with purification by column chromatography (SiO₂, hexane:ethyl acetate 1:5) affording DR33 as a white solid (13 mg, 0.04 mmol, 20%).

m.p. 176-178° C. (lit. m.p. 175° C.); δ_{H} (400 MHz, d₆-DMSO) 3.75 (3H, s, OMe), 3.79 (3H, s, OMe), 3.85 (3H, s, OMe), 3.94 (3H, s, OMe), 6.57 (1H, s, H-3), 7.06 (1H, d, J 8.6 Hz, H-5′), 7.14 (1H, s, H-8), 7.42 (1H, d, J 2.1 Hz, H-2′), 7.49 (1H, dd, J 8.6 and 2.1 Hz, H-6′), 9.41 (1H, s, OH); δ_{C} (100 MHz, d₆-DMSO) 56.0 (CH₃), 56.7 (CH₃), 61.2 (CH₃), 62.1 (CH₃), 97.5 (CH), 106.3 (CH), 112.3 (CH), 113.0 (CH), 118.3 (CH), 123.5 (C), 140.0 (C), 147.0 (C), 150.9 (C), 151.8 (C), 154.2 (C), 157.6 (C), 160.8 (C), 175.8 (C); vₘₐₓ (KBr disc) 3100, 1630, 1590, 1530, 1420, 1260, 1120 cm⁻¹; m/z (FAB) 359 [MH⁺, 100%]; (Found C, 64.0; H, 5.3. C₁₉H₁₈O₇ requires C, 63.7; H, 5.1%).

2-(3′-Hydroxy-4′-methoxyphenyl)-6,7,8-trimethoxy-4H-chromen-4-one (DR36).

The flavone DR36 was obtained following protocol H using DR27 (100 mg, 0.28 mmol) and potassium cyanide (180 mg, 2.80 mmol) in ethanol (5 cm³), with purification by column chromatography (SiO₂, hexane:ethyl acetate 1:10) and recrystallisation from hexane:ethyl acetate affording DR36 as a pale yellow solid (32 mg, 0.09 mmol, 32%).

m.p. 199-200° C.; δ_{H} (400 MHz, CDCl₃) 3.97 (3H, s, OMe), 3.99 (3H, s, OMe), 4.05 (3H, s, OMe), 4.10 (3H, s, OMe), 5.95 (1H, s, OH), 6.72 (1H, s, H-3), 6.98 (1H, d, J 8.4 Hz, H-5′), 7.40 (1H, s, H-5), 7.52 (1H, d, J 8.4 and 2.2 Hz, H-6′), 7.53 (1H, d, J 2.2 Hz, H-2′); δ_{C} (100 MHz, CDCl₃) δ6.5 (CH₃), 56.7 (CH₃), 61.9 (CH₃), 62.5 (CH₃), 100.4 (CH), 106.2 (CH), 111.2 (CH), 112.7 (CH), 119.3 (CH), 120.2 (C), 125.5 (C), 142.5 (C), 146.2 (C), 146.4 (C), 147.7 (C), 149.8 (C), 151.5 (C), 163.2 (C), 178.1 (C); vₘₐₓ (KBr disc) 3100, 1570, 1470, 1430, 1390, 1260, 1120 cm⁻¹; m/z (FAB) 359 [MH⁺, 50%]; (Found C, 64.0; H, 4.9. C₁₉H₁₈O₇ requires C, 63.7; H, 5.1%).

(E)-3-(3″-Fluoro-4″-methoxyphenyl)-2-methyl-1-(3′,4′,5′-trimethoxyphenyl)-2-propen-1-one (DR5).

General procedure: A solution of 3,4,5-trimethoxypropiophenone (4 mmol), substituted benzaldehyde (4 mmol), piperidine (0.8 mL) and acetic acid (0.4 ml) in ethanol (80 mL), was heated to reflux using a Soxhlet apparatus with a thimble containing activated molecular sieves to remove water from the solvent. After 4-7 days, the solvent was removed in vacuo and the product purified by column chromatography.

The chalcone DR5 was obtained following protocol A using 3,4,5-trimethoxypropiophenone (0.36 g, 1.61 mmol), 3-fluoro-4-methoxybenzaldehyde (0.25 g, 1.61 mmol), piperidine (0.30 cm³) and acetic acid (0.15 cm³) in ethanol (3.5 cm³). The mixture was heated at reflux under argon for 4 days. Purification by column chromatography (SiO₂, hexane:ethyl acetate 3:1) afforded DR5 as a white solid (0.36 g, 1.00 mmol, 62%).

m.p. 84-86° C.; δ_{H} (300 MHz, CDCl₃) 2.26 (3H, s, Me), 3.89 (6H, s, OMe), 3.92 (6H, s, OMe), 6.98 (2H, s, H-2′, H-6′), 6.99 (1H, d, J 8.6 Hz, H-S″), 7.08 (1H, s, H-3), 7.17 (1H, dd, J 8.6 and 2.0 Hz, H-6″) , 7.24 (1H, dd, J Hz, 13.0 and 2.0H-2″) ; c (75 MHz, CDCl₃) 15.1 (CH₃), 56.6 (CH₃), 56.7 (CH₃), 61.3 (CH₃), 107.5 (CH), 113.5 (CH, d, J 2.0 Hz), 117.6 (CH, d, J 15.0 Hz), 127.0 (CH, d, J 5.0 Hz), 129.2 (C, d, J 5.0 Hz), 136.1 (C), 133.8 (C), 140.3 (CH), 141.8 (C), 148.4 (C, d, J 15.0 Hz), 152.4 (C, d, J 247.0 Hz), 153.2 (C), 198.7 (C); δ_{F} (200 MHz, CDCl₃); vₘₐₓ (KBr disc) 1580, 1520, 1420, 1340, 1240, 1130 cm⁻¹; m/z (FAB) 361 [MH⁺, 100%], 191 (80); (Found C, 66.8; H, 5.6; F, 5.6. C₂₀H₂₁O₅F requires C, 66.7; H, 5.9; F, 5.3%).

3-Fluoro-4-methoxybenzaldehyde.

The method adopted was that of Diana and co-workers (Diana 1989). A stirring solution of 2-fluoroanisole (4.46 cm³, 39.7 mmol) and hexamethylenetetramine (5.57 g, 39.7 mmol) in trifluoroacetic acid (35 cm³) was heated at reflux under argon overnight. On cooling to room temperature the solvent was evaporated in vacuo and the crude residue dissolved in dichloromethane (75 cm³). The mixture was washed with an aqueous solution of sodium hydrogen carbonate (2×30 cm³), dried over anhydrous magnesium sulfate, filtered and evaporated in vacuo to afford 3-fluoro-4-methoxybenzaldehyde as a pale yellow solid (3.32 g, 21.6 mmol, 54%).

m.p. 30-31° C. (English et al., 1940 reported m.p. 29-30° C.); δ_{H} (300 MHz, CDCl₃) 3.98 (3H, s, OMe), 7.08 (1H, t, J 8.0 Hz, H-5), 7.60 (2H, m H-2, H-6), 9.87 (1H, d, J 5.0 Hz, CHO); δ_{C} (75 MHz, CDCl₃) 56.7 (CH₃), 113.1 (CH), 115.9 (CH, d, J 15.0 Hz), 128.6 (CH, d, J 3.0 Hz), 130.4 (C, J 5.0 Hz), 152.5 (C, d, J 250.0 Hz), 153.4 (C, J 15.0 Hz), 190.2 (CH); vₘₐₓ (KBr disc) 1690, 1610, 1570, 1440, 1290, 1120 cm⁻¹; m/z (FAB) 153 [M⁺, 100%], 223 (100); (Found C, 62.3; H, 4.6. C₈H₇O₂F requires C, 62.0; H, 4.5%).

(E)-3-(3″, 5″-Difluoro-4″-methoxyphenyl)-2-methyl-1-(3′, 4′,5′-trimethoxyphenyl)-2-propen-1-one (DR6).

The chalcone DR6 was obtained following the general method using 3,4,5-trimethoxypropiophenone (0.35 g, 1.56 mmol), 3,5-difluoro-4-methoxybenzaldehyde (0.27 g, 1.56 mmol), piperidine (0.40 cm³) and acetic acid (0.20 cm³) in ethanol (2.0 cm³). The mixture was heated at reflux under argon for 4 days. Purification by column chromatography (SiO₂, hexane:ethyl acetate 3:1) afforded DR6 as a colourless solid (0.11 g, 0.29 mmol, 19%).

δ_{H} (300 MHz, CDCl₃) 2.30 (3H, s, Me), 3.90 (6H, s, OMe), 3.95 (3H, s, OMe), 4.00 (3H, s, OMe), 6.95-7.05 (5H, m, H-3, H-2′, H-6′, H-2″, H-6″) ; δ_{C} (75 MHz, CDCl₃) 15.2 (CH₃), 56.7 (CH₃), 61.3 (CH₃), 62.2 (CH₃), 107.5 (CH), 113.8 (CH, dd, J 13.0 and 5.0 Hz), 130.6 (C, t, J 7.0 Hz), 133.2 (C), 136.9 (C, t, J 13.0 Hz), 138.0 (C), 138.2 (CH, split, J 3.0 Hz), 142.2 (C), 153.3 (C), 155.6 (C, dd, J 244.0 and 7.0 Hz), 198.3 (C); δ_{F} (200 MHz, CDCl₃); vₘₐₓ (KBr disc) 1640, 1590, 1520, 1420, 1330, 1130 cm⁻¹; m/z (FAB) 379 [MH⁺, 100%]; (Found C, 63.7; H, 5.2; F, 9.7. C₂₀H₂₀O₅F₂ requires C, 63.5; H, 5.3; F, 10.0%).

3,5-Difluoro-4-methoxybenzaldehyde.

To a stirring solution of 3,5-difluoro-4-hydroxybenzaldehyde (1.52 g, 9.6 mmol) in dimethylformamide (7.5 cm³) was added potassium carbonate (1.99 g, 14.4 mmol) and iodomethane (0.70 cm³, 11.5 mmol). The mixture was stirred at room temperature under argon overnight, diluted with dichloromethane (50 cm³) and washed with an aqueous solution of sodium hydrogen carbonate (2×25 cm³). The organic fraction was dried over anhydrous magnesium sulfate, filtered and evaporated in vacuo to afford 3,5-difluoro-4-methoxybenzaldehyde as a white solid (1.20 g, 7.0 mmol, 73%).

m.p. 37-38° C. (Songca 1997 reported m.p. 37-38° C.); δ_{H} (300 MHz, CDCl₃) 4.12 (3H, s, OMe), 7.43 (2H, m, H-2, H-6), 9.82 (1H, s, CHO); δ_{C} (75 MHz, CDCl₃) 62.0 (CH₃), 113.9 (CH, dd, J 20.0 and 3.0 Hz), 130.6 (C, t, J 10.0 Hz), 142.2 (C, t, J 20.0 Hz), 157.7 (C, dd, J 250.0 and 10.0 Hz), 189.1 (CH); Vₘₐₓ (KBr disc) 1700, 1620, 1590, 1520, 1450, 1390, 1340 cm⁻¹; m/z (EI) 172 [M⁺, 100%]; (Found C, 55.7; H, 3.5; F, 21.8. C₈H₆O₂F₂ requires C, 55.8; H, 3.5; F, 22.1%).

Disodium 3′-phosphate salt of (E)-1-(3′-Hydroxy-4′-methoxyphenyl)-3-(3″,4″,5″-trimethoxyphenyl)prop-1-en-3-one (SD174a).

According to the method of Perich and Jones (Perich 1988), 1H-tetrazole (408 mg, 5.82 mmol) was added in one portion to a stirred solution of chalcone 1-(3″-hydroxy-4″-methoxyphenyl)-3-(3′, 4′, 5′-trimethoxyphenyl)-1-propen-3-one (583 mg, 1.69 mmol) and di-tert-butyl N,N-diethylphosphoramidite (0.43 cm³, 1.54 mmol) in dry THF (5 cm³) and stirred for 20 min at room temperature under an atmosphere of nitrogen. The mixture was then cooled down to −78° C. and a solution of m-CPBA (57% w/w, 631 mg, 2.08 mmol) in dry DCM (2 cm³) was added. After stirring for 10 min at room temperature, a 10% aqueous solution of sodium bisulfite (4 cm³) was added and the mixture stirred for a further 15 min. The aqueous mixture was then extracted with diethyl ether (50 cm³) and the ethereal layer washed with a 10% aqueous solution of sodium bisulfite (2×20 cm³), a 5% aqueous solution of sodium bicarbonate (2×20 cm³), a 0.5 M aqueous solution of sodium hydroxide (2×20 cm³) and finally water (20 cm³). The ethereal layer was then dried over anhydrous magnesium sulfate, filtered and evaporated in vacuo to give the corresponding di-tert-butyl phosphate triether (770 mg, 1.43 mmol, 85%); m/z (FAB) 539 (M+H)⁺, 40%), 425 (30); a solution of 10 M hydrochloric acid:1,4-dioxane (1:1, 10 cm³) was added to the residue and the reaction was allowed to stand at room temperature for 1 h. The solvent was evaporated under reduced pressure (temperature<45° C.) and water (15 cm³) was added to the residue. The resultant precipitate was collected and washed with chloroform (20 cm³) to give the 3′-phosphoryl chalcone SD173a as a yellow oil (390 mg, 0.92 mmol, 54%). δ_{H} (300 MHz, d₆-DMSO) 3.07 (3H, s, OMe), 3.12 (3H, s, OMe), 3.15 (6H, s, OMe), 6.33 (1H, d, J 8.8 Hz, H-5′), 6.61 (2H, s, H-2, H-6), 6.75 (1H, dd, J 4.4, 8.8 Hz, H-6′), 6.88-7.00 (3H, m, H-1, H-2, H-2′); δₚ (81 MHz, d₆-DMSO)-0.17; m/z (FAB) 425 [(M+H)⁺, 100%], 424 (M+, 50); chalcone SD173a (108 mg, 0.25 mmol) was dissolved in a 1:1 mixture of methanol:water (4 cm³) and two drops of a 35% w/v aqueous ammonia solution were added. The mixture was applied to a Dowex 50W-X8 cation-exchange column (10 cm³, Na⁺), the column was eluted with a 1:1 mixture of methanol:water (30 cm³) and the eluent was concentrated to give disodium 3′-phosphoryl chalcone SD174a as a bright yellow powder (87 mg, 0.19 mmol, 76%); m.p. 160° C. (dec.); vₘₐₓ (KBr disc) 2700-3200, 1650, 1580, 1510, 1430-1470, 1270, 1130, 990 cm⁻¹; λₘₐₓ (EtOH) 206.7 (log ε 4.41) and 358.9 nm (log ε 4.01); δ_{H} (300 MHz, d₆-DMSO) 3.07 (3H, s, OMe), 3.12 (3H, s, OMe), 3.15 (6H, s, OMe), 6.33 (1H, d, J 8.8 Hz, H-5′), 6.61 (2H, s, H-2″, H-6), 6.75 (1H, dd, J 2.4, 8.8 Hz, H-6′), 6.88-7.00 (3H, m, H-1, H-2, H-2′); δₚ (81 MHz, d₆-DMSO)-87.2; [found (FAB): (M+H)⁺, 469.0630. C₁₉H₂₀O₉PNa₂ requires 469.0641]; m/z (FAB) 491 [(M+Na)⁺, 60%], 469 [(M+H)⁺, 60], 329 (50), 176 (100).

Disodium 3′-phosphate salt of (E)-1-(3′-Hydroxy-4′-methoxyphenyl)-2-methyl-3-(3″,4″,5″-trimethoxyphenyl)prop-1-en-3-one (SD174b).

1H-Tetrazole (237 mg, 3.38 mmol) was added to a stirred solution of chalcone DR4 (970 mg, 2.71 mmol) and di-tert-butyl N,N-diethylphosphoramidite (0.75 cm³, 2.69 mmol) in dry DCM (10 cm³) and stirred for 20 min at room temperature under an atmosphere of nitrogen. The reaction mixture was then cooled down to −78° C. and m-CPBA (57% w/w, 945 mg, 3.12 mmol, dried over anhydrous magnesium sulfate) in dry DCM (5 cm³) was added. After stirring for 10 min at room temperature, a 10% aqueous solution of sodium bisulfite (8 cm³) was added and the mixture was stirred for a further 15 min. The aqueous mixture was extracted with diethyl ether (30 cm³) and the ethereal layer was washed successively with a 10% aqueous solution of sodium bisulfite (10 cm³), a 5% aqueous solution of sodium bicarbonate (10 cm³), a 0.5 M aqueous solution of sodium hydroxide (10 cm³) and finally with water (10 cm³). The solvent was removed in vacuo from the organic extract, the residue was redissolved in 10 M hydrochloric acid:1,4-dioxan (1:1, 10 cm³) and then the mixture was left to stand at room temperature for 2 hours. The solvents were removed and water (20 cm³) was added to the residue. The resultant precipitate was collected by filtration, washed with water (20 cm³) and dissolved in a 1:1 mixture of methanol:water and 2 drops of a 35% w/v aqueous solution of ammonia were added. The mixture was applied to a Dowex 50W-X8 cation-exchange resin column (15 cm³, Na⁺), where the column was eluted with water (30 cm³), then concentrated to give disodium 3′-phosphoryl chalcone SD174b as a yellow powder (40 mg, 0.083 mmol, 39%); m.p. 170° C. (dec.); vₘₐₓ (KBr disc) 2700-3200, 1640, 1600, 1580, 1520, 1410, 1340, 1280, 1240, 1120, 990 cm⁻¹; λₘₐₓ (EtOH) 208.6 (log ε 4.52) and 326.2 nm (log ε 4.12); δ_{H} (300 MHz, D₂O) 2.20 (3H, s, Me), 3.82 (3H, s, OMe), 3.84 (6H, s, OMe), 3.86 (3H, s, OMe), 6.98 (2H, s, H-2, H-6″), 7.02 (1H, d, J 8.5 Hz, H-5′), 7.14 (2H, m, H-2′, H-6′), 7.60 (1H, brs, H-2); δ_{C} (75 MHz, D₂O) 15.2 (CH₃), 57.3 (CH₃), 57.6 (CH₃), 62.4 (CH₃), 99.9 (C), 108.8 (CH), 113.7 (CH), 123.4 (CH), 126.8 (CH), 129.6 (C), 135.9 (C), 141.4 (C), 144.4 (C), 146.7 (CH), 152.4 (C), 153.5 (C), 204.1 (C); 8p (81 MHz, D₂O)-87.0; [found (FAB) (M+H)⁺, 483.0812. C₂₀H₂₂O₉PNa₂ requires 483.0798]; m/z (FAB) 505 [(M+Na)⁺, 60%], 483 [(M+H)⁺, 75], 391 (30), 329 (30), 289 (40), 176 (100), 136 (50).

Biological Activity

The compounds of the present invention have been studied to ascertain their effectiveness as anti-cancer agents.

The compounds of the present invention have been tested for their tubulin inhibitory properties, and the results are presented in Tables 1-8, where they are compared with combretastatin A-4. The compounds of the present invention have, for convenience, been split into groups based on structural features of the compounds. The corrected values are scaled by a factor of 5 to compensate for the fact that the experimental IC50 for combretastatin A4 is lower than is often quoted in the literature.

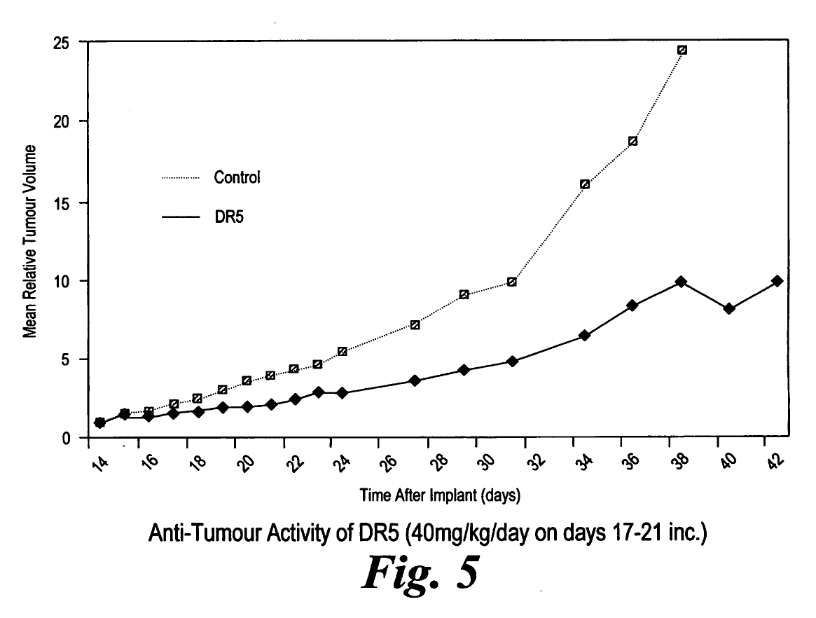

Compound DR5 was tested for in vivo as follows. Groups of 5 nude mice were implanted s.c. in the flank with H460 human non small cell lung cells. Tumour growth was monitored by caliper measurement. Treatment was started once tumour growth had been verified. Control mice were treated with vehicle alone (arachis oil). Treatment was given daily for 5 days at 8 mg/kg/day (days 17-21). Tumour volumes were calculated relative to the tumour volume on the first day of treatment (day 17 after implantation). Weight loss and general condition were monitored for the duration of the study. The experiments showed necrosis in H460 cancer cells treated with compound DR5 24 hours after treatment with 0.75 MTD. There were no adverse side effects on healthy surrounding tissue. The results of this experiment are shown in FIG. 5.

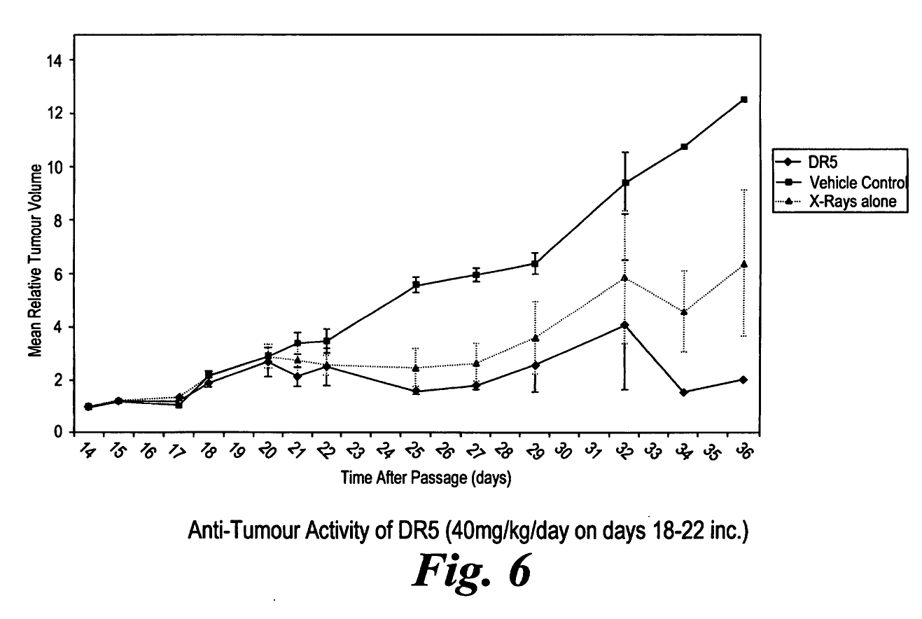

Further improvement in the potency of DRA 212 was seen in an experiment in which where H460 xenograft mice were treated with X-Rays alone or were concomitantly treated with X-Rays and DRA 212 (FIG. 6). Whilst X-Ray treatment was effective immediately after treatment, fresh tumour growth became evident by 36 days. In the X-Ray plus DR5 treated group, there was some initial increase in tumour volume between days 27 and 32, though this was followed by subsequent decrease to a steady baseline at day 34.

The compounds have been further tested for their performance in colchicine competition assays, and the results tabulated in Tables 9 to 13.

TABLE 1
Tubulin assembly inhibitory properties of
3,4,5-trimethoxyphenylchalcones.

| | | IC₅₀ μM |
|---|---|---|
| Drug | IC₅₀ μM (original) | (corrected) |
| DR2 | 1.2 | 6 |
| DR3 | 12 | 60 |
| DR5 | 0.7 | 3.5 |
| DR6 | 2.4 | 12 |
| Combretastatin A-4 | 0.4 | 2.0 |

TABLE 2
Tubulin assembly inhibitory properties of water-
soluble prodrugs (chalcones).

| | | IC₅₀ μM |
|---|---|---|
| Drug | IC₅₀ μM (original) | (corrected) |
| DR55 | 39 | >100 |
| DR56 | 3.1 | 16 |
| combretastatin A-4 | 0.4 | 2.0 |

TABLE 3
Tubulin assembly inhibitory properties of
α-methoxychalcones.

| | | IC₅₀ μM |
|---|---|---|
| Drug | IC₅₀ μM (original) | (corrected) |
| DR13 | 0.51 | 2.6 |
| DR14 | 0.47 | 2.4 |
| DR15 | 1.7 | 8.5 |
| combretastatin A-4 | 0.4 | 2.0 |

TABLE 4
Tubulin assembly inhibitory properties of
2,3,4-trimethoxyphenylchalcones.

| | | IC₅₀ μM |
|---|---|---|
| Drug | IC₅₀ μM (original) | (corrected) |
| DR8 | 0.45 | 2.3 |
| DR9 | 7.9 | 40 |
| DR10 | 31 | >100 |
| combretastatin A-4 | 0.4 | 2.0 |

TABLE 5
Tubulin assembly inhibitory properties of
aurones.

| | | IC₅₀ μM |
|---|---|---|
| Drug | IC₅₀ μM (original) | (corrected) |
| DR23 | >50 | >100 |
| DR24 | >50 | >100 |
| DR27 | 22 | >100 |
| DR28 | >50 | >100 |
| combretastatin A-4 | 0.4 | 2.0 |

TABLE 6
Tubulin assembly inhibitory properties of
flavones.

| | | IC₅₀ μM |
|---|---|---|
| Drug | IC₅₀ μM (original) | (corrected) |
| DR33 | >50 | >100 |
| DR34 | >50 | >100 |
| DR36 | 25 | >100 |
| DR37 | >50 | >100 |
| combretastatin A-4 | 0.4 | 2.0 |

TABLE 7
Tubulin assembly inhibitory properties of
indanones and indanols.

| | | IC₅₀ μM |
|---|---|---|
| Drug | IC₅₀ μM (original) | (corrected) |
| DR57 | 1.9 | 9.5 |
| DR58 | 9.8 | 49 |
| DR59 | 4.0 | 20 |
| DR60 | >50 | >100 |
| combretastatin A-4 | 0.4 | 2.0 |

TABLE 8
Tubulin assembly inhibitory properties of
catechol-chalcones.

| | | IC₅₀ μM |
|---|---|---|
| Drug | IC₅₀ μM (original) | (corrected) |
| DR31 | >50 | >100 |
| combretastatin A-4 | 0.4 | 2.0 |

TABLE 9
Colchicine competition properties of chalcones.

Drug:Protein Ratio

| Drug | 10:1 | 1:1 |
|---|---|---|
| DR5 | 6 | 14 |
| DR6 | 25 | 33 |
| combretastatin A-4 | 8 | 17 |

TABLE 10
Colchicine competition properties of water-
soluble prodrugs.

Drug:Protein Ratio

| Drug | 10:1 | 1:1 |
|---|---|---|
| DR55 | 83 | 100 |
| DR56 | 12 | 100 |
| combretastatin A-4 | 8 | 17 |

TABLE 11
Colchicine competition properties of α-
alkoxychalcones.

Drug:Protein Ratio

| Drug | 10:1 | 1:1 |
|---|---|---|
| DR13 | 5 | 12 |
| DR14 | 8 | 22 |
| DR15 | 41 | 59 |
| combretastatin A-4 | 8 | 17 |

TABLE 12
Colchicine competition properties of aurones
and flavones.

Drug:Protein Ratio

| Drug | 10:1 | 1:1 |
|---|---|---|
| DR27 | 59 | 78 |
| DR36 | 43 | 100 |
| combretastatin A-4 | 8 | 17 |

TABLE 13
Colchicine competition properties of indanones.

Drug:Protein Ratio

| Drug | 10:1 | 1:1 |
|---|---|---|
| DR57 | 15 | 54 |
| DR59 | 61 | 100 |
| combretastatin A-4 | 8 | 17 |

Tables 14 and 15 show the results of tubulin assembly assays and flow cytometry studies on selected compounds of the present invention.

Tubulin Assembly Assay

TABLE 14
shows the IC(TA)₅₀ values calculated for selected
compounds of the present invention.

| Drug | Structure | IC(TA)₅₀ |
|---|---|---|
| MW71 | 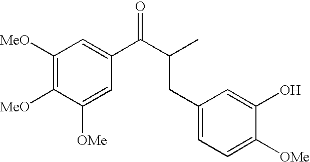 | 4 μM |
| MW70 | 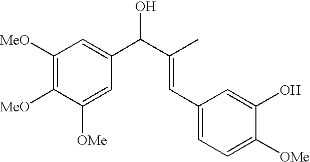 | >10 μM |
| MW74 | 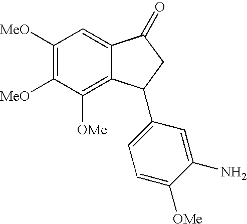 | ˜10 μM |
| MW68 | 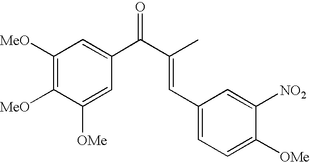 | >10 μM |
| MW82 | 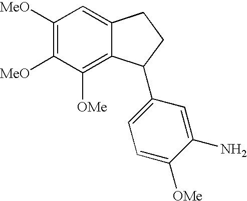 | >10 μM |

Flow Cytometry

TABLE 15
percentage of cells in the three phases of the cell cycle calculated by the computer program for the
selected drugs.

| | | % Cells | | | |
|---|---|---|---|---|---|
| Drug | Structure | G₀-G₁ | S-phase | G₂-M | Debris |
| Control | | 55.05 | 32.87 | 12.08 | |
| MW65 | 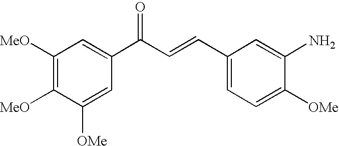 | 48.30 | 33.18 | 18.52 | 14.10 |
| MW68 | 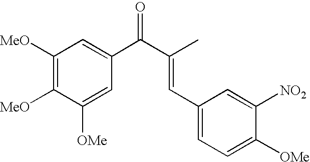 | 36.35 | 35.36 | 28.29 | 11.27 |
| MW70 | 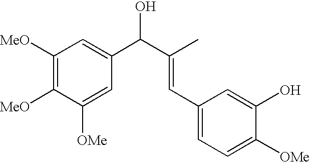 | 43.50 | 32.80 | 23.70 | 15.27 |
| MW71 | 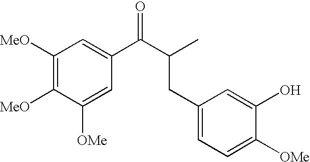 | 35.84 | 36.09 | 28.08 | 19.31 |
| MW74 | 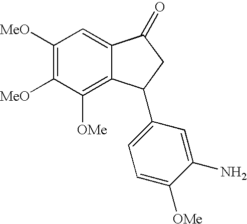 | 37.14 | 33.76 | 29.10 | 12.72 |
| MW82 | 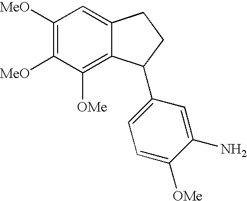 | 40.40 | 36.26 | 23.34 | 18.58 |

### REFERENCES

The references mentioned herein are all expressly incorporated by reference.
1. Arizona Board of Regents, U.S. Pat. No. 4,996,237.
2. Ducki et al, Bioorg. Med. Chem. Lett., 1998, 8, 1051.
3. Zhao et al, Eur. J. Nuc. Medicine, 1999, 26, 231.
4. Aleksandrzak et al, Anti-Cancer Drugs, 1998, 9, 545.
5. Lee, K.-H.; Chen, K.; Kuo, S.-C., U.S. Pat. No. 6,071,930.
6. Edwards, M. L.; Stemerick, D. M.; Sunkara, S. P., EP 0 288 794 A2.
7. Clark, D.; Frankmoelle, W.; Houze, J.; Jaen, J. C.; Medina, J. C.; WO00/35865A2.
8. Klein, L. L. et al, J. Med. Chem., 1991, 984.
9. Beutler, J. A. et al, J. Med. Chem., 1998, 2333.
10. Huang, L. et al, J. Natural Products, 1998, 61, 446-450.
11. D. D. Pratt and R. Robinson, J. Chem. Soc., 1925, 127, 173.
12. W. J. Horton and G. Thompson, J. Am. Chem. Soc., 1954, 76, 1909.
13. P. K. Mahajan, Par, Yogender, Anand and Shalu, Indian J. Chem. Sect. B., 1996, 35, 333.
14. D. J. Abraham, P. E. Kennedy, A. S. Mehanna, D. C. Patwa and F. L. Williams, J. Med. Chem., 1984, 27, 967.
15. G. D. Diana, D. Cutcliffe, R. C. Oglesby, M. J. Otto, J. P. Mallamo, V. Akullian and M. A. McKinlay, J. Med. Chem., 1989, 32, 450.
16. J. English, J. F. Mead and C. Niemann, J. Am. Chem. Soc., 1940, 62, 352.
17. S. P. Songca, R. Bonnett and C. Maes, S. Afr. J. Chem., 1997, 50, 40.
18. J. W. Perich, R. B. Johns, Synthesis, 1988, 142.
19. C. Giordano, G. Castaldi, F. Casagrande and L. Abis, Tetrahedron Lett., 1982, 23, 1385.
20. R. S. Varma and M. Varma, Tetrahedron Lett., 1992, 33, 5937.
21. D. M. Fitzgerald, J. F. O'Sullivan, E. M. Philbin and T. S. Wheeler, J. Chem. Soc., 195, 860.

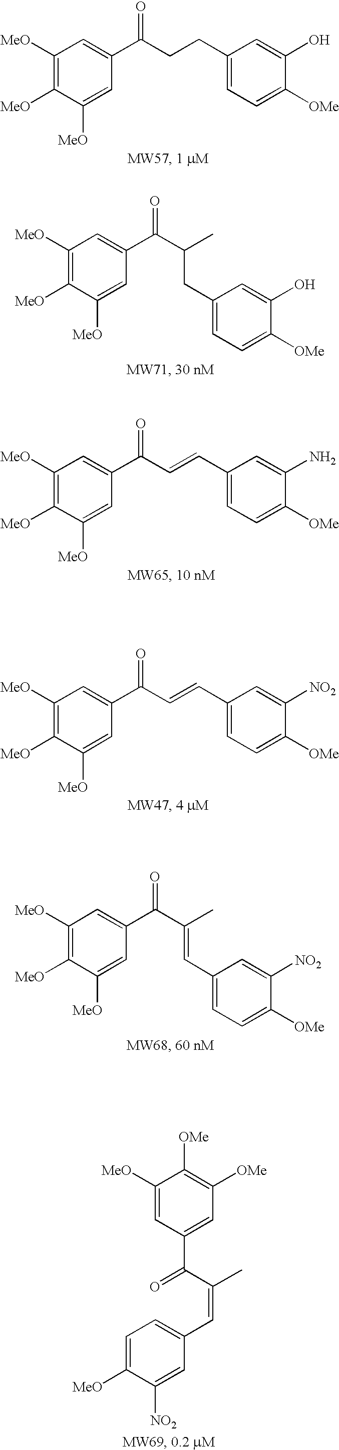

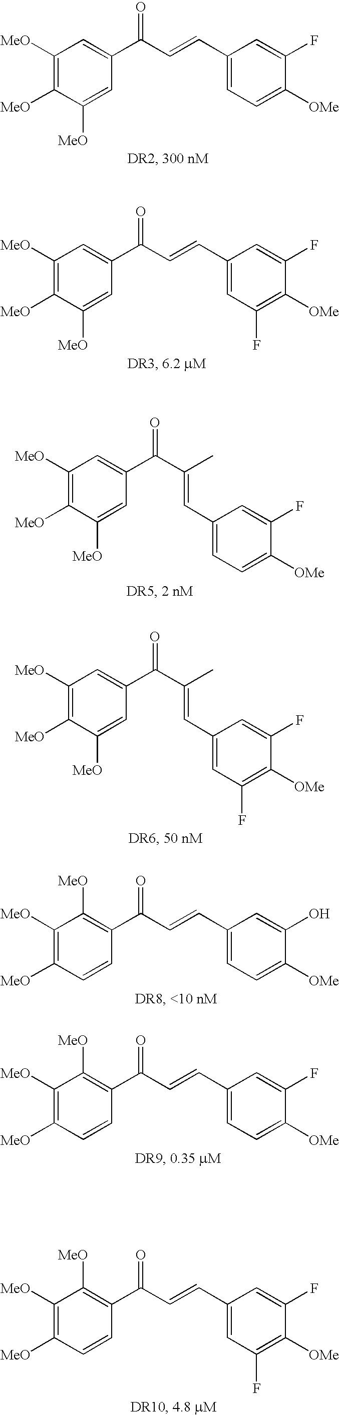

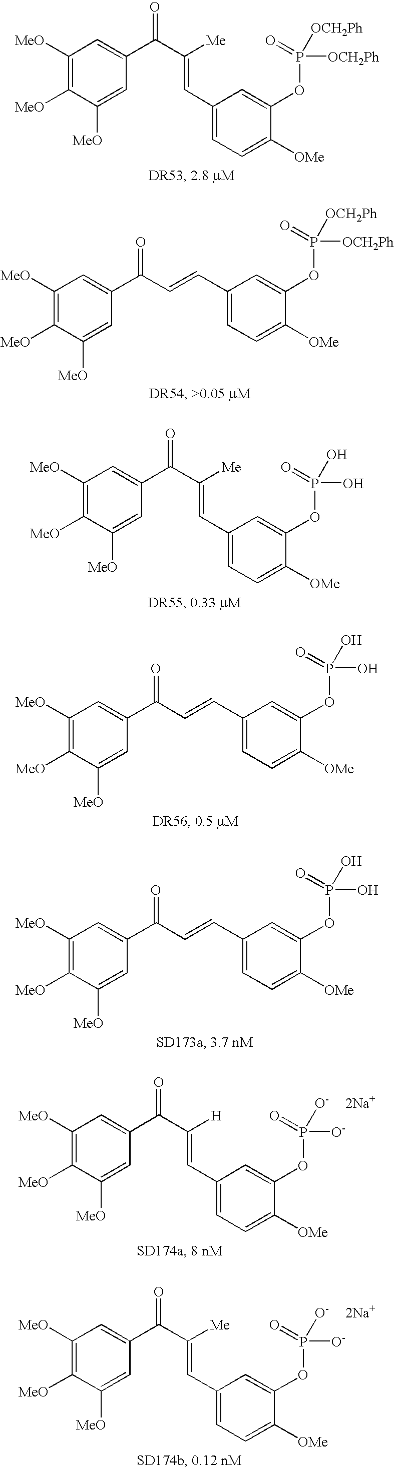

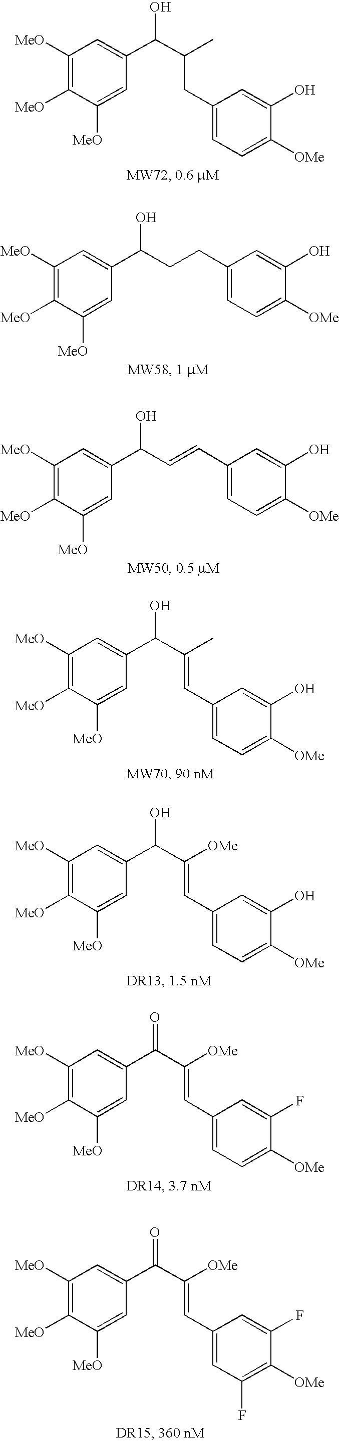

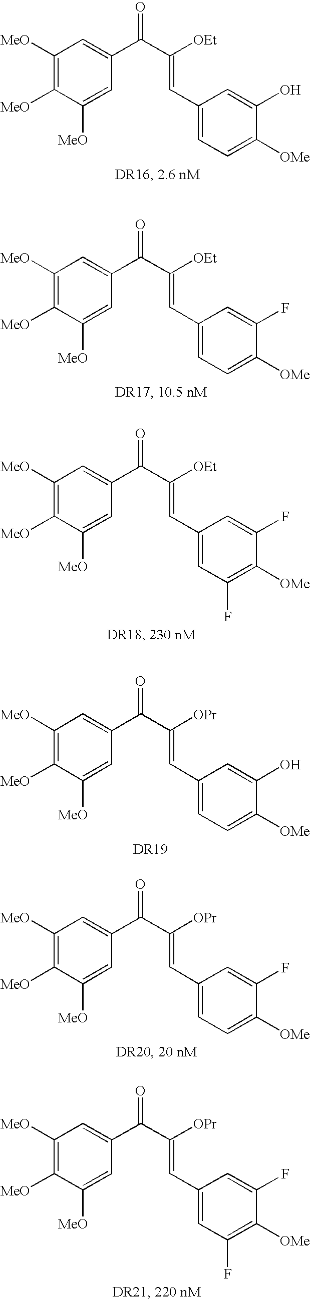

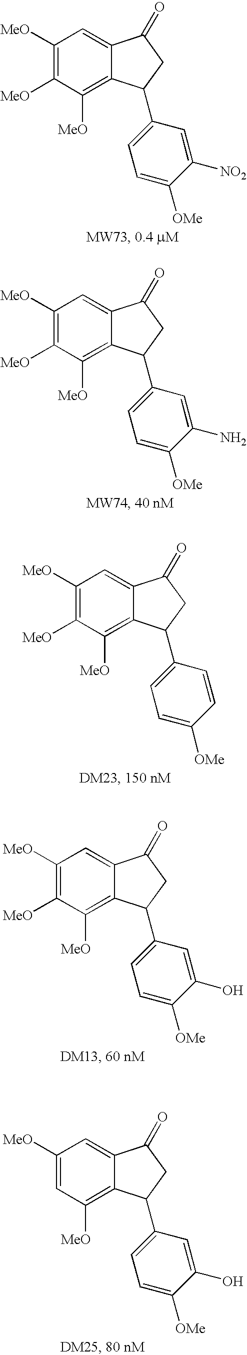

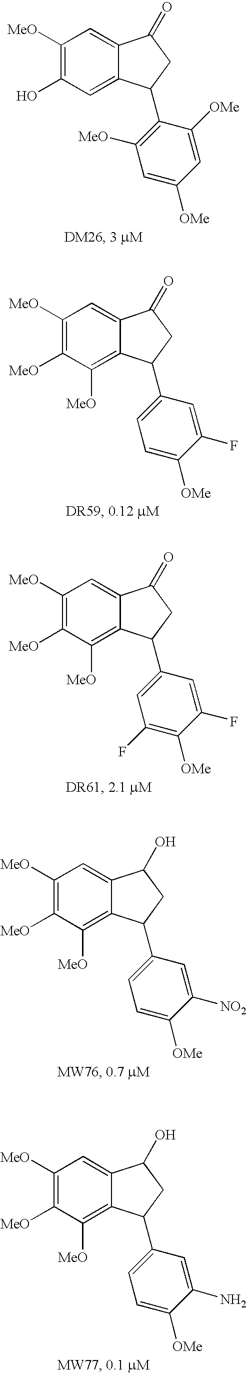

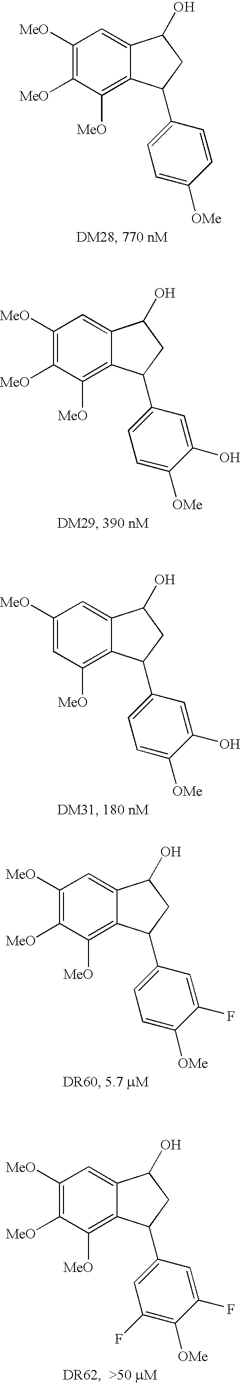

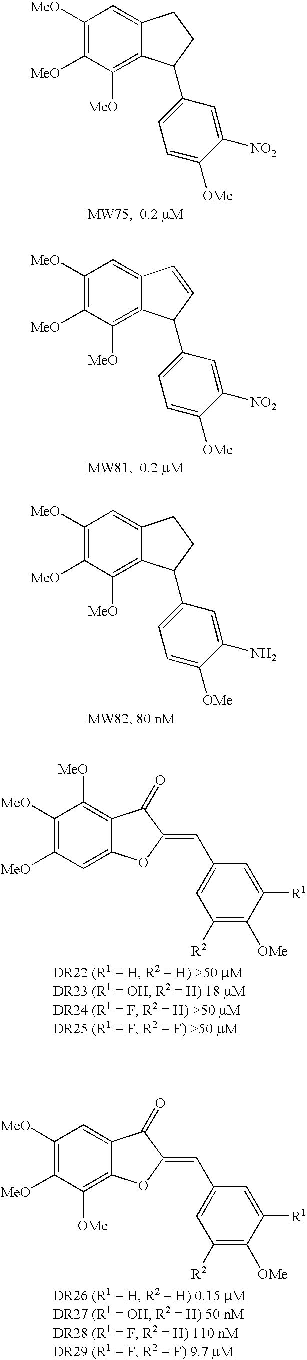

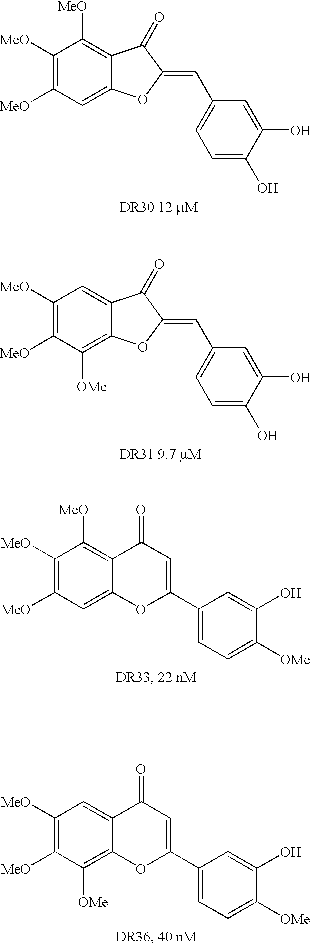

1. A compound represented by formula I: 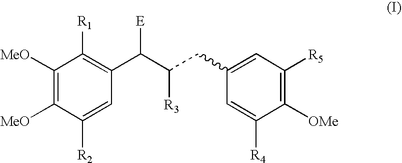
wherein:
E represents an oxo (═O) or a hydroxyl (—OH);
the dashed line indicates that a single or double bond may be present;
the zig-zag line indicates that the compound can be either the E or Z isomer;
R₃ is H, alkyl, CH₂NH₂, CH₂NHalkyl, CH₂OH, CH₂N(alkyl)₂, CH₂NH(C═O)alkyl, CH₂NH(C═O)aryl; and
R₄ is H, halogen, NH(alkyl), N(alkyl)₂, NH(C═O)alkyl, NH(C═O)aryl, or a Boc-ester group represented by: 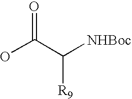
wherein R₉ is alkyl, CH₂Ph where Ph is a substituted or substituted phenyl group, or an amino acid side chain; and further wherein:
when E is an oxo (═O) group and the dashed line represents a single bond,
R₁ is H; R₂ is alkoxy; R₄ is H; and R₅ is OH; or
when E is an oxo (═O) group and the dashed line represents a double bond,
R₁ is H; R₂ is alkoxy; R₄ is H or halogen; and
R₅ is H or halogen; or
R₁ is H; R₂ is alkoxy; R₄ is H; and R₅ is NH₂, NO₂, halogen or OPO₃(R₆)₂; where R₆ is H, CH₂Ph or a metal cation; or
R₁ is alkoxy; R₂ is H; R₄ is H or halogen; and
R₅ is halogen or OH; or
when E is a hydroxyl (—OH) group and the dashed line represents a single or double bond,
R₁ is H; R₂ is alkoxy; R₃ is methyl; R₄ is H; and R₅ is OH;
or a salt or derivative thereof.

2. The compound of claim 1, wherein the compound is a compound represented by formula I where:
E is an oxo (═O) group; the dashed line represents a single bond; R₁ is H; R₂ is OMe; R₃ is H; R₄ is H; and R₅ is OH (MW57);
E is an oxo (═O) group; the dashed line represents a single bond; R₁ is H; R₂ is OMe; R₃ is Me; R₄ is H; and R₅ is OH (MW71);
E is an oxo (═O) group; the dashed line represents a double bond; R₁ is H; R₂ is OMe; R₃ is H; R₄ is H; and R₅ is NH₂ (MW65);
E is an oxo (═O) group; the dashed line represents a double bond; R₁ is H; R₂ is OMe; R₃ is H; R₄ is H; and R₅ is NO₂ (MW47);
E is an oxo (═O) group; the dashed line represents a double bond; the compound is the E isomer; R₁ is H; R₂ is OMe; R₃ is Me; R₄ is H; and R₅ is NO₂ (MW68);
E is an oxo (═O) group; the dashed line represents a double bond; the compound is the Z isomer; R₁ is H; R₂ is OMe; R₃ is Me; R₄ is H; and R₅ is NO₂ (MW69);
E is an oxo (═O) group; the dashed line represent a double bond; R₁ is H; R₂ is OMe; R₃ is H; R₄ is H; and R₅ is F (DR2);
E is an oxo (═O) group; the dashed line represent a double bond; R₁ is H; R₂ is OMe; R₃ is H; R₄ is F; and R₅ is F (DR3);
E is an oxo (═O) group; the dashed line represent a double bond; R₁ is H; R₂ is OMe; R₃ is Me; R₄ is H; and R₅ is F (DR5);
E is an oxo (═O) group; the dashed line represent a double bond; R₁ is H; R₂ is OMe; R₃ is Me; R₄ is F; and R₅ is F (DR6);
E is an oxo (═O) group; the dashed line represent a double bond; R₁ is OMe; R₂ is H; R₃ is H; R₄ is H; and R₅ is OH (DR8);
E is an oxo (═O) group; the dashed line represent a double bond; R₁ is OMe; R₂ is H; R₃ is H; R₄ is H; and R₅ is F (DR9);
E is an oxo (═O) group; the dashed line represent a double bond; R₁ is OMe; R₂ is H; R₃ is H; R₄ is F; and R₅ is F (DR10);
E is an oxo (═O) group; the dashed line represent a double bond; R₁ is H; R₂ is OMe; R₃ is Me; R₄ is H; and R₅ is OPO₃(R₆)₂ wherein R₆ is CH₂Ph (DR53);
E is an oxo (═O) group; the dashed line represent a double bond; R₁ is H; R₂ is OMe; R₃ is H; R₄ is H; and R₅ is OPO₃(R₆)₂ wherein R₆ is CH₂Ph (DR54);
E is an oxo (═O) group; the dashed line represent a double bond; R₁ is H; R₂ is OMe; R₃ is Me; R₄ is H; and R₅ is OPO₃(R₆)₂ wherein R₆ is H (DR55);
E is an oxo (═O) group; the dashed line represent a double bond; R₁ is H; R₂ is OMe; R₃ is H; R₄ is H; and R₅ is OPO₃(R₆)₂ wherein R₆ is H (DR56);
E is an oxo (═O) group; the dashed line represent a double bond; R₁ is H; R₂ is OMe; R₃ is H; R₄ is H; and R₅ is OPO₃(R₆)₂ wherein R₆ is H (SD173a);
E is an oxo (═O) group; the dashed line represent a double bond; R₁ is H; R₂ is OMe; R₃ is H; R₄ is H; and R₅ is OPO₃(R₆)₂ wherein R₆ is Na (SD174a);
E is an oxo (═O) group; the dashed line represent a double bond; R₁ is H; R₂ is OMe; R₃ is Me; R₄ is H; and R₅ is OPO₃(R₆)₂ wherein R₆ is Na (SD174b);
E is a hydroxyl (—OH) group; the dashed line represents a single bond; R₁ is H; R₂ is OMe; R₃ is Me; R₄ is H; and R₅ is OH (MW72);
E is a hydroxyl (—OH) group; the dashed line represents a single bond; R₁ is H; R₂ is OMe; R₃ is H; R₄ is H; and R₅ is OH (MW58);
E is a hydroxyl (—OH) group; the dashed line represents a double bond; R₁ is H; R₂ is OMe; R₃ is H; R₄ is H; and R₅ is OH (MW50);
E is a hydroxyl (—OH) group; the dashed line represents a double bond; R₁ is H; R₂ is OMe; R₃ is Me; R₄ is H; and R₅ is OH (MW70).

3. A compound represented by formula Ia: 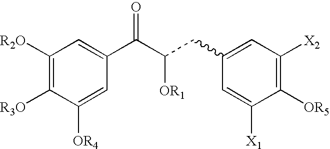
wherein:
the dashed line indicates that a single or double bond may be present;
the zig-zag line indicates that the compound can be either the E or Z isomer;
R₁ is alkyl; R₂, R₃, R₄ and R₅ are independently selected from H or alkyl; X₁ and X₂ are independently selected from H, OH, nitro, amino, aryl, heteroaryl, alkyl, alkoxy, CHO, COR, halogen, haloalkyl, NH₂, NHR, NRR′, SR, CONH₂, CONHR, CONHRR′, O—P═O(OR)₂, O-aryl, O-heteroaryl, O-ester, R and R′ being substituted or unsubstituted, branched or unbranched C₁₋₁₀ alkyl groups or aryl or heteroaryl groups, or a Boc-ester group represented by: 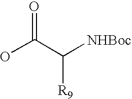
wherein R₉ is alkyl, CH₂Ph where Ph is a substituted or substituted phenyl group, or an amino acid side chain;
or a salt or derivative thereof.

4. The compound of claim 3, wherein the compound is a compound represented by formula Ia where:
the dashed line represent a double bond; R₁ is Me; R₂, R₃ and R₄ are Me; R₅ is Me; X₁ is H; and X₂ is OH (DR13); or
the dashed line represent a double bond; R₁ is Me; R₂, R₃ and R₄ are Me; R₅ is Me; X₁ is H; and X₂ is F (DR14); or
the dashed line represent a double bond; R₁ is Me; R₂, R₃ and R₄ are Me; R₅ is Me; X₁ and X₂ are F (DR15); or
the dashed line represent a double bond; R₁ is Et; R₂, R₃ and R₄ are Me; R₅ is Me; X₁ is H; and X₂ is OH (DR16); or
the dashed line represent a double bond; R₁ is Et; R₂, R₃ and R₄ are Me; R₅ is Me; X₁ is H; and X₂ is F (DR17); or
the dashed line represent a double bond; R₁ is Et; R₂, R₃ and R₄ are Me; R₅ is Me; X₁ and X₂ are F (DR18); or
the dashed line represent a double bond; R₁ is Pr; R₂, R₃ and R₄ are Me; R₅ is Me; X₁ is H; and X₂ is OH (DR19); or
the dashed line represent a double bond; R₁ is Pr; R₂, R₃ and R₄ are Me; R₅ is Me; X₁ is H; and X₂ is F (DR20); or
the dashed line represent a double bond; R₁ is Pr; R₂, R₃ and R₄ are Me; R₅ is Me; X₁ is F; and X₂ is F (DR21).

5. A compound represented by formula II: 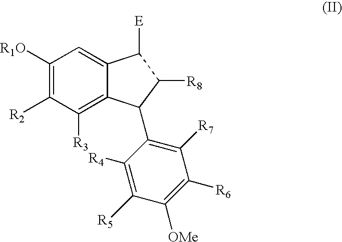
wherein:
E represents an oxo (═O), hydroxyl (—OH) or a hydrogen atom;
the dashed line in the structure indicates that a single or double bond may be present; and
R₈ is hydrogen, alkyl, aryl, CH₂NH₂, CH₂NHalkyl or CH₂N(alkyl)₂; and wherein:
when E is an oxo (═O) group and the dashed line represents a single bond,
R₁ is alkyl or H; R₂ is alkoxy or H; R₃ is alkoxy or H; and
R₄ is H; R₅ is H, O(P═O) (OR)₂ or Boc-ester;
R₆ is NO₂, NH₂, H, OH, halogen, NHMe, NHMe₂, NH(C═O)alkyl or NH(C═O)aryl; and R₇ is H; or
R₄ is H; R₅ is halogen, O(P═O) (OR)₂ or Boc-ester;
R₆ is OH, halogen, NHMe, NHMe₂, NH(C═O)alkyl or NH(C═O)aryl; and R₇ is H; or
R₄ is alkoxy; R₅ is H, O(P═O) (OR)₂ or Boc-ester;
R₆ is H, NHMe, NHMe₂, NH(C═O)alkyl or NH(C═O)aryl; and R₇ is alkoxy; or
when E is a hydroxyl (—OH) group and the dashed line represents a single bond,
R₁ is alkyl; R₂ is H or alkoxy; R₃ is alkoxy; R₄ is H; R₅ is alkoxy, halogen, O(P═O) (OR)₂ or Boc-ester;
R₆ is H, NO₂, NH₂, OH, halogen, NHMe, NHMe₂, NH(C═O)alkyl or NH(C═O)aryl; and R₇ is H; or
when E is a hydrogen atom and the dashed line represents a double bond,
R₁ is Me; R₂ is alkoxy; R₃ is alkoxy; R₄ is H; R₅ is H, O(P═O) (OR)₂ or Boc-ester;
R₆ is NO₂, NH₂, NHMe, NHMe₂, NH(C═O)alkyl or NH(C═O)aryl; and R₇ is H;
wherein the Boc-ester is a group represented by: 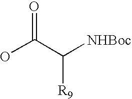
wherein R₉ is alkyl, CH₂Ph where Ph is a substituted or substituted phenyl group, or an amino acid side chain; or
a compound represented by structural formula IIa: 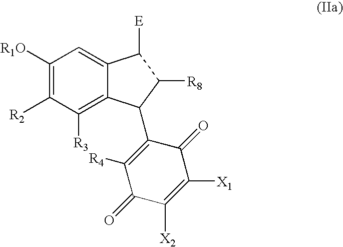
wherein:
E, R₁, R₂, R₃, R₄ and R₈ are as defined above; and
X₁ and X₂ are independently selected from H, OH, nitro, amino, aryl, heteroaryl, alkyl, alkoxy, CHO, COR, halogen, haloalkyl, NH₂, NHR, NRR′, SR, CONH₂, CONHR, CONHRR′, O-aryl, O-heteroaryl or O-ester, R and R′ being substituted or unsubstituted, branched or unbranched C₁₋₁₀ alkyl groups or aryl or heteroaryl groups;
or a salt or derivative of compounds II or IIa.

6. The compound of claim 5, wherein when the compound is a compound represented by formula II where:
E is an oxo (═O) group; the dashed line represents a single bond; R₁ is Me; R₂ is OMe; R₃ is OMe; R₄ is H; R₅ is H; R₆ is NO₂; R₇ is H; R8 is H (MW73); or
E is an oxo (═O) group; the dashed line represents a single bond; R₁ is Me; R₂ is OMe; R₃ is OMe; R₄ is H; R₅ is H; R₆ is NH₂; and R₇ is H; R8 is H (MW74); or
E is an oxo (═O) group; the dashed line represents a single bond; R₁ is Me; R₂ is OMe; R₃ is OMe; R₄ is H; R₅ is H; R₆ is H; and R₇ is H; R8 is H (DM23); or
E is an oxo (═O) group; the dashed line represents a single bond; R₁ is Me; R₂ is OMe; R₃ is OMe; R₄ is H; R₅ is H; R₆ is OH; and R₇ is H; R8 is H (DM13); or
E is an oxo (═O) group; the dashed line represents a single bond; R₁ is Me; R₂ is H; R₃ is OMe; R₄ is H; R₅ is H; R₆ is OH; and R₇ is H; R8 is H (DM25); or
E is an oxo (═O) group; the dashed line represents a single bond; R₁ is Me; R₂ is OH; R₃ is H; R₄ is OMe; R₅ is H; R₆ is H; and R₇ is OMe; R8 is H (DM26); or
E is an oxo (═O) group; the dashed line represents a single bond; R₁ is Me; R₂ is OMe; R₃ is OMe; R₄ is H; R₅ is H; R₆ is F; and R₇ is H; R8 is H (DR59); or
E is an oxo (═O) group; the dashed line represents a single bond; R₁ is Me; R₂ is OMe; R₃ is OMe; R₄ is H; R₅ is F; R₆ is F; and R₇ is H; R8 is H (DR61); or
E is a hydroxyl (—OH) group; the dashed line represents a single bond; R₁ is Me; R₂ is OMe; R₃ is OMe; R₄ is H; R₅ is H; R₆ is NO₂; R₇ is H; R8 is H (MW76); or
E is a hydroxyl (—OH) group; the dashed line represents a single bond; R₁ is Me; R₂ is OMe; R₃ is OMe; R₄ is H; R₅ is H; R₆ is NH₂; and R₇ is H; R8 is H (MW77); or
E is a hydroxyl (—OH) group; the dashed line represents a single bond; R₁ is Me; R₂ is OMe; R₃ is OMe; R₄ is H; R₅ is H; R₆ is H; and R₇ is H; R8 is H (DM28); or
E is a hydroxyl (—OH) group; the dashed line represents a single bond; R₁ is Me; R₂ is OMe; R₃ is OMe; R₄ is H; R₅ is H; R₆ is OH; and R₇ is H; R8 is H (DM29); or
E is a hydroxyl (—OH) group; the dashed line represents a single bond; R₁ is Me; R₂ is H; R₃ is OMe; R₄ is H; R₅ is H;
R₆ is OH; and R₇ is H; R8 is H (DM31); or
E is a hydroxyl (—OH) group; the dashed line represents a single bond; R₁ is Me; R₂ is OMe; R₃ is OMe; R₄ is H; R₅ is H; R₆ is F; and R₇ is H; R8 is H (DR60); or
E is a hydroxyl (—OH) group; the dashed line represents a single bond; R₁ is Me; R₂ is OMe; R₃ is OMe; R₄ is H; R₅ is F; R₆ is F; and R₇ is H; R8 is H (DR62); or
E is a hydrogen atom; the dashed line represents a single bond; R₁ is Me; R₂ is OMe; R₃ is OMe; R₄ is H; R₅ is H; R₆ is NO₂; and R₇ is H; R8 is H (MW75); or
E is a hydrogen atom; the dashed line represents a double bond; R₁ is Me; R₂ is OMe; R₃ is OMe; R₄ is H; R₅ is H; R₆ is NO₂; and R₇ is H; R8 is H (MW81); or
E is a hydrogen atom; the dashed line represents a single bond; R₁ is Me; R₂ is OMe; R₃ is OMe; R₄ is H; R₅ is H; R₆ is NH₂; and R₇ is H; R8 is H (MW82).

7. A compound represented by formula III: 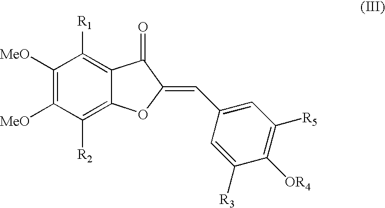
wherein:
R₁ is H or alkoxy; R₂ is H or alkoxy; R₃ is H or halogen; R₄ is H or alkyl; and R₅ is H, OH, halogen, O(P═O) (OR)₂, R being a substituted or unsubstituted, branched or unbranched C₁₋₁₀ alkyl group or aryl or heteroaryl groups, or a Boc-ester group represented by: 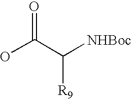
wherein R₉ is alkyl, CH₂Ph where Ph is a substituted or substituted phenyl group, or an amino acid side chain;
or a salt or derivative thereof.

8. The compound of claim 7, wherein the compound is a compound represented by formula III where:
R₁ is OMe; R₂ is H; R₃ is H; R₄ is Me; R₅ is H (DR22); or
R₁ is OMe; R₂ is H; R₃ is H; R₄ is Me; R₅ is OH (DR23); or
R₁ is OMe; R₂ is H; R₃ is H; R₄ is Me; R₅ is F (DR24); or
R₁ is OMe; R₂ is H; R₃ is F; R₄ is Me; R₅ is F (DR25); or
R₁ is H; R₂ is OMe; R₃ is H; R₄ is Me; R₅ is H (DR26); or
R₁ is H; R₂ is OMe; R₃ is H; R₄ is Me; R₅ is OH (DR27); or
R₁ is H; R₂ is OMe; R₃ is H; R₄ is Me; R₅ is F (DR28); or
R₁ is H; R₂ is OMe; R₃ is F; R₄ is Me; R₅ is F (DR29); or
R₁ is H; R₂ is OMe; R₃ is H; R₄ is H; R₅ is OH (DR31).

9. A compound represented by formula IV: 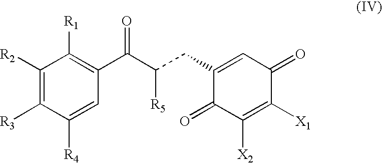
wherein:
the dashed line indicates that a single or double bond may be present;
the zig-zag line indicates that the compound can be either the E or Z isomer; and
R₁, R₂, R₃ and R₄ are independently selected from H or alkoxy;
R₅ is hydrogen, alkyl, alkoxy or O-aryl; and
X₁ and X₂ are independently selected from H, OH, nitro, amino, aryl, heteroaryl, alkyl, alkoxy, CHO, COR, halogen, haloalkyl, NH₂, NHR, NRR′, SR, CONH₂, CONHR, CONHRR′, O-aryl, O-heteroaryl or O-ester, R and R′ being substituted or unsubstituted, branched or unbranched C₁₋₁₀ alkyl groups or aryl or heteroaryl groups;
or a salt or derivative thereof.

10. The compound of claim 9, wherein the compound is a compound represented by formula IV where the dashed line represents a double bond; R₁ is H; R₂ is OMe; R₃ is OMe; R₄ is OMe, X₁ is OMe, and X₂ is H.

11. The compound of claim 1, wherein said alkyl substituent is a substituted or unsubstituted methyl or ethyl group.

12. The compound of claim 1, wherein said alkoxy substituent is a substituted or unsubstituted methoxy, ethoxy or propoxy group.

13. The compound of claim 1, wherein said halogen group is a fluorine group.

14. The compound of claim 1, wherein the salt or derivative is a salt, an ester, a free acid or base, a hydrate, a prodrug or the compound linked to a coupling partner.

15. The compound of claim 14, wherein the salt is a sodium phosphate salt, a sodium salt, a potassium salt, a lithium salt, a magnesium salt, a calcium salt, a manganese salt, a zinc salt, a salt with an ammonium cation selected from imidazole, morpholine, piperazine, piperidine, pyrazole, pyridine, adenosine, cinchonine, glucosamine, quinine, quinidine, tetracycline and verapamil.

16. The compound of claim 14, wherein the ester is a Boc-ester, a hemisuccinic acid ester, a phosphate ester, a sulphate ester or a selenate ester.

17. A pharmaceutical composition comprising a compound of claim 1, or a salt or derivative thereof, and a carrier.

18. (Cancelled)

19. A method for the treatment of cancer or a condition involving abnormal proliferation of vasculature in a patient in need of said treatment by administering a therapeutically effective amount of a compound of claim 1.

20. The method of claim 19, wherein the condition is diabetic retinopathy, psoriasis or endometriosis.

21. A compound represented by structural formula V: 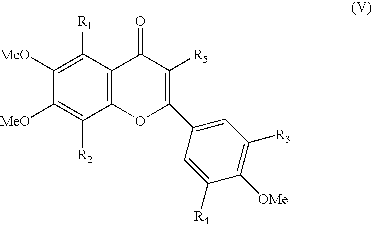
wherein:
R₁ or R₂ is alkoxy and the other is H;
R₃ and R₄ are different and are hydrogen, halogen, OH, O(P═O) (OR)₂ or Boc-ester;
R₅ is aryl, alkyl or O-alkyl;
wherein the Boc-ester group is represented by: 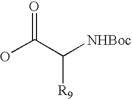
wherein R₉ is alkyl, CH₂Ph where Ph is a substituted or substituted phenyl group, or an amino acid side chain; or
a compound represented by structural formula Va in which: 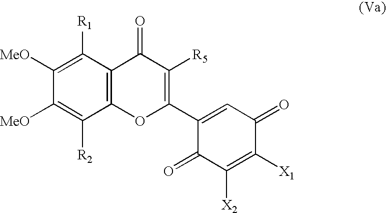
wherein:
R₁, R₂ and R₅ are defined as above;
X₁ and X₂ are independently selected from H, OH, nitro, amino, aryl, heteroaryl, alkyl, alkoxy, CHO, COR, halogen, haloalkyl, NH₂, NHR, NRR′, SR, CONH₂, CONHR, CONHRR′, O-aryl, O-heteroaryl or O-ester; or
or a salt or derivative of compounds V or Va.

22. The compound of claim 21, wherein the compound is a compound represented by formula V where:
R₁ is OMe; R₂ is H; R₃ is OH; and R₄ is H; or
R₁ is OMe; R₂ is H; R₃ is F; and R₄ is H; or
R₁ is H; R₂ is OMe; R₃ is OH; and R₄ is H; or
R₁ is OMe; R₂ is H; R₃ is F; and R₄ is H.

23. A method for the treatment of cancer or a condition involving abnormal proliferation of vasculature in a patient in need of said treatment by administering a therapeutically effective amount of a compound of claim 21.

24. The method of claim 23, wherein the condition is diabetic retinopathy, psoriasis or endometriosis.
